(19) United States
(12) Patent Application Publication
(10) Pub. No.: US 2024/0423976 A1
(43) Pub. Date: Dec. 26, 2024

(54) COMPOSITIONS AND METHODS FOR TREATING THERAPEUTIC-INDUCED TOXICITIES

(71) Applicant: THE BOARD OF REGENTS OF THE UNIVERSITY OF OKLAHOMA, Norman, OK (US)

(72) Inventors: Vibhuti AGRAHARI, Edmond, OK (US); Neeraj S. THAKUR, Oklahoma City, OK (US)

(73) Assignee: THE BOARD OF REGENTS OF THE UNIVERSITY OF OKLAHOMA, Norman, OK (US)

(21) Appl. No.: 18/749,968

(22) Filed: Jun. 21, 2024

Related U.S. Application Data
(60) Provisional application No. 63/509,823, filed on Jun. 23, 2023.
(60) Provisional application No. 63/652,458, filed on May 28, 2024.

(51) Int. Cl. A61K 31/495; A61K 9/06; A61K 31/05; A61K 47/10; A61K 47/32; A61K 47/34

(52) U.S. Cl. CPC A61K 31/495; A61K 9/06; A61K 31/05; A61K 47/10; A61K 47/32; A61K 47/34

(57) ABSTRACT

A method and composition for mitigating or reducing drug-induced ototoxicity or drug-induced hearing loss in a subject in need of such treatment, comprising: providing a drug delivery system which releases a therapeutic drug upon exposure to reactive oxygen species, wherein the drug delivery system comprises a thermoresponsive hydrogel containing therapeutic drug-loaded crosslinked hybrid nanoparticles and provides sustained therapeutic drug release. The crosslinked hybrid nanoparticles within which the therapeutic drug is loaded may comprise 10,12-Pentacosadiynoic acid (PCDA) and polypropylene sulfide-polyethylene glycol monomethyl ether (PPS-mPEG). The drug-induced ototoxicity or hearing loss may be caused by cisplatin or other platinum-based drugs.

### CROSS REFERENCE TO RELATED APPLICATIONS

This application claims benefit of U.S. Provisional Application Ser. No. 63/509,823 filed Jun. 23, 2023, and U.S. Provisional Application Ser. No. 63/652,458, filed May 28, 2024. The entire contents of both of the above-referenced patent applications are hereby expressly incorporated herein by reference.

### STATEMENT REGARDING FEDERALLY SPONSORED RESEARCH OR DEVELOPMENT

This invention was made with government support under grant number DK122028 awarded by the National Institutes of Health. The government has certain rights in the invention.

### BACKGROUND

Cisplatin-based chemotherapies are commonly used to treat several types of cancers, but are frequently responsible for permanent inner ear hair cell damage leading to ototoxicity and hearing loss (HL). The reported rate of cisplatin-induced hearing loss (CIHL) ranges between 11% and 97%, with an average incidence of 62%. Hearing loss resulting from chemotherapeutic induced ototoxicity is irreversible. Treatment-induced ototoxicity has been reported for different categories of drugs such as aminoglycosides, vancomycin, macrolides, and loop diuretics. Though the ototoxicity of loop diuretics, macrolide antibiotics, and quinine is reported to be reversible when the treatment is stopped, cases have been reported for irreversible HL. However, platinum drugs, especially cisplatin, and aminoglycosides like kanamycin, showed irreversible damage to the outer hair cells (OHCs) and inner ear hair cells (IHCs) of cochlea, thereby affecting patients' quality of life significantly due to hearing impairment or permanent hearing loss. Treatment-induced hearing loss (TIHL) or hearing impairments can lead to considerable follow-up costs estimated to be $300,000 per adult who acquired hearing impairment due to ototoxic medication. Considering ototoxicity as a well-known obstacle of the several therapeutic classes of drugs, a prophylactic cure or early treatment would be valuable in protecting the IHCs and OHCs. The mechanism involves accumulation in the cochlea, oxidative stress, apoptosis, and inflammation leading to cell death and hearing loss. To mitigate ototoxicity, protective strategies like otoprotective agents and careful monitoring of patients' hearing can be developed. For instance, many cytoprotective molecules have been investigated as cytoprotective agents against Cisplatin-induced ototoxicity (CIO), including Amifostine, Sodium Thiosulfate (STS), Dexamethasone, N-acetylcysteine, Ebselen, Flunarizine, Agmatine, Honokiol, and Allicin. Recently, the FDA approved STS IV for prevention in children, however, no treatments are available to prevent or reverse CIO in adults. Moreover, the patient-centric compliance of the administration of any molecule still requires significant research and development. Therefore, developing effective formulations for local delivery to the inner ear is one of the challenges to preventing treatment-induced ototoxicity and hearing loss due to ototoxic medications.

Flunarizine (FL), a ‘T-type’ calcium channel blocker, has been studied for its potential to mitigate cisplatin-induced cell cytotoxicity and ototoxicity. However, this effect was not mediated by the modulation of intracellular calcium levels. The cytoprotective effect of FL against CIO in HEI-OC1 cells by downregulation of NF-κB via Nrf2/HO-1 activation was reported, which results in reduced pro-inflammatory cytokine production. HK, a natural compound from the magnolia tree, has been investigated for its potential protective effects against CIO. Its antioxidant properties may help reduce reactive oxygen species (ROS) production, protecting the inner ear from oxidative damage. Its anti-inflammatory properties may mitigate the inflammatory response, potentially reducing cisplatin-induced damage. Mechanistically, it has been reported to activate sirtuin 3 (SIRT-3), a mitochondrial deacetylase that is responsible for ROS detoxification. It may also be explored as part of combination therapy with other molecules to get promising cytoprotective effects against drug-induced ototoxicity and associated hearing loss.

### BRIEF DESCRIPTION OF THE DRAWINGS

Aspects of several embodiments of the present disclosure are illustrated in the appended drawings. The appended drawings only illustrate several embodiments and are not intended to be limiting of the scope of the inventive concepts disclosed herein. The patent or application file contains at least one drawing executed in color. Copies of this patent or patent application publication with color drawing(s) will be provided by the Office upon request and payment of the necessary fee.

FIG. 1 shows a schematic presentation of the development of stimuli-responsive nanoparticle-based formulation and the corresponding mechanism of action of drug delivery and inhibition of ROS-associated apoptotic cell death and subsequent hearing loss.

FIG. 2 shows DoE-CCD optimization graphs for the synthesis of BUC-PPS-mPEG₂₀₀₀-NPs. The panels (i), (ii), and (iii), represent the encapsulation efficiency (EE %), Drug loading (DL %), and prediction profiler, respectively.

FIG. 3 shows DoE-CCD optimization graphs for the synthesis of DLT-PPS-mPEG₂₀₀₀-NPs. The panels (i), (ii), and (iii), represent the encapsulation efficiency (EE %), Drug loading (DL %), and prediction profiler, respectively.

FIG. 4 shows size and morphology analyses of BUC-PPS-mPEG₂₀₀₀-NPs. The upper left panel shows the size distribution of BUC-PPS-mPEG₂₀₀₀-NPs analyzed by DLS. The upper right panel shows the size distribution of BUC-PPS-mPEG₂₀₀₀-NPs analyzed by nanoparticle tracking analyzer (NTA). The lower left panel presents the visual BUC-PPS-mPEG₂₀₀₀-NPs under NTA Nanosight instrument. The lower right panel shows a TEM image of BUC-PPS-mPEG₂₀₀₀-NPs showing spherical and intact shape (scale 100 nm).

FIG. 5 shows size and morphology analyses of DLT-PPS-mPEG₂₀₀₀-NPs. The upper left panel shows the size distribution of DLT-PPS-mPEG₂₀₀₀-NPs analyzed by DLS. The upper right panel shows the size distribution of DLT-PPS-mPEG₂₀₀₀-NPs analyzed by NTA. The lower left panel presents the visual DLT-PPS-mPEG₂₀₀₀-NPs under NTA Nanosight instrument. The lower right panel shows a TEM image of DLT-PPS-mPEG₂₀₀₀-NPs showing spherical and intact shape (scale 100 nm).

FIG. 6 shows results of a cellular uptake and intracellular distribution study of NPs using Coumarin-6-PPS-mPEG₂₀₀₀-NPs. The upper panel shows the time required for significant cell internalization: The graph shows the fluorescence intensities of the extracted media after the lysis of the cells that were incubated with C-6-loaded NPs for different lengths of time (1, 2, 3, 4, 5, and 6 h) (λex=460±40 nm, λem=528±20 nm). The cells showed significant cellular internalization after 4 h of incubation compared to 1 h. The highest internalization was observed at 6 h of incubation. However, no significant difference in fluorescence intensity between 5 and 6 h suggests >90% cellular uptake within 5 h. Therefore, the peak incubation time for cellular internalization of PPS-mPEG₂₀₀₀-NPs resides between 5-6 h. These results indicate that PPS-mPEG₂₀₀₀-NPs were able to successfully internalize and distributed into HEI-OC1 cells. (The groups were compared using one-way ANOVA using Sidik's multiple comparison post-hoc test. Asterisks: ****p<0.0001; **p≤0.005; *p≤0.05; ns=not significant). The lower panel shows a cartoon of the molecular mechanism of internalization. Clathrin-mediated internalization is a plausible molecular mechanism of cellular internalization of C-6-PPS-mPEG₂₀₀₀-NPs. The depicted image describes the all steps involved in this process which include (i) receptor recognition and attachment, (ii) formation, stabilization, and release of clathrin-coated vesicle, (iii) uncoating, (iv) endosome recognition and fusion, and (v) release in the cytoplasm.

FIG. 7 shows results of fluorescence microscopy used to confirm the cellular uptake and intracellular distribution in the experiments of FIG. 6. The fluorescence microscopy images of the cells incubated with C-6-PPS-mPEG₂₀₀₀-NPs at different time intervals (1, 2, 3, 4, 5, and 6 h from topmost row to lowermost row). Four images are shown in each row corresponding to each time interval: (a) HOECHST 33342 stained nuclei (λex=358 nm, λem=461 nm), (b) Coumarin-6 fluorescence in the respective cells (λex=480 nm, λem=502 nm), (c) cells under transmitted light, (d) an image with results of a, b and c merged. The fluorescence of C-6 in the cells was observed at different time intervals. (Scale 90 μm). These images indicate that PPS-mPEG₂₀₀₀-NPs were able to successfully internalize and become distributed into HEI-OC1 cells.

FIG. 8 shows methods of synthesis and characterization of NanoSensoGel formulation. (a) A photograph showing the effect of temperature on the NanoSensoGel formulation. The formulation's sol state converts to gel state at/above 33° C., while below 33° C. the formulation remains in the sol state. (b) A schematic presentation of components of the NanoSensoGel and the appearance of sol-gel states at varying temperatures. At lower temperature, the gel is present in a flowing sol state while at a higher temperature the formulation components are arranged in the micellar structure and form a nonflowing gel state (created using Biorender.com). (c) SEM images of the NanoSensoGel. (i and ii) SEM images of hydrogel were kept below 30° C. at sol state then snap-freezed using liquid nitrogen at −196° C. and lyophilized subsequently. The sample observed under the SEM shows the un-arranged pattern in the structure (scale 10 μm). (iii and iv) SEM images of hydrogel were kept at 34° C. gel state then snap-freezed and lyophilized subsequently. The sample observed under the SEM showed a well-arranged honeycomb-type pattern in the structure (scale 10 μm).

FIG. 9 shows (d-i) a pictorial representation of a well-insert that was used for the drug release study from the hydrogel (created using Biorender.com), and (d-ii) the pictures captured after setting up the release experiments. In (d-ii) the upper photo shows a side view of wells containing NanoSensoGel formulation in the insert, and the lower photo shows the upper view of the NanoSensoGel and free drugs containing gel. (e-i) is a graph which compares the percent release of BUC from NanoSensoGel and free BUC-Gel. (e-ii) is a graph which compares the percent release of DLT from NanoSensoGel and free DLT-Gel. (f) is a graph which shows the cytoprotective effect of NanoSensoGel compared with free BUC/DLT-Gel, free BUC/DLT-PPS-mPEG₂₀₀₀-NPs, and free BUC/DLT solution after the cell (HEI-OC1) exposed to CisPt (The data were analyzed using one-way ANOVA using Šidák's multiple comparison post-hoc test. Asterisks: ****P<0.0001, ns=not significant).

FIG. 10 is a schematic presentation for (a) the synthesis of FL-cHy-NPs and HK-cHy-NPs used herein, and (b) of the arrangement of the PCDA, PPS, and encapsulated drugs (FL or HK) and subsequent crosslinking during the synthesis of FL-cHy-NPs and HK-cHy-NPs.

FIG. 11A shows actual vs. predicted plots of particle sizes and PDI responses after performing all experiments and running the Standard Least Square model for FL-cHy-NPs with emphasis on effective screening. (b) Plots for HK-cHy-NPs. The upper panel shows the responses for each NP preparation presented as Particle Size (nm). The lower panel shows the responses for each NP preparation presented as PDI. In each response plot, the black dots represent the response values obtained after performing the experiments, the horizontal line represents the null hypothesis and the slanted line represents an alternative hypothesis. The shaded area around the slanted line represents the 95% confidence region. RMSE shows the average measured values of difference between the predicted and actual values. RSq (R²) values under each response plot shows how close the data fitted to the regression line (alternative hypothesis).

FIG. 11B shows actual vs. predicted plots of EE (%) and Ld (%) responses after performing all experiments and running the Standard Least Square model for FL-cHy-NPs with emphasis on effective screening. (b) Plots for HK-cHy-NPs. The upper panel shows the responses for each NP preparation presented as EE (%). The lower panel shows the responses for each NP preparation presented as EE (%). In each response plot, the black dots represent the response values obtained after performing the experiments, the horizontal line represents the null hypothesis and the slanted line represents an alternative hypothesis. The shaded area around the slanted line represents the 95% confidence region. RMSE shows the average measured values of difference between the predicted and actual values. RSq (R²) values under each response plot shows how close the data fitted to the regression line (alternative hypothesis).

FIG. 11C shows actual vs. predicted plots of particle sizes and PDI responses after performing all experiments and running the Standard Least Square model for HK-cHy-NPs with emphasis on effective screening. The upper panel shows the responses for each NP preparation presented as Particle Size (nm). The lower panel shows the responses for each NP preparation presented as PDI. In each response plot, the black dots represent the response values obtained after performing the experiments, the horizontal line represents the null hypothesis and the slanted line represents an alternative hypothesis. The shaded area around the slanted line represents the 95% confidence region. RMSE shows the average measured values of difference between the predicted and actual values. RSq (R²) values under each response plot shows how close the data fitted to the regression line (alternative hypothesis).

FIG. 11D shows actual vs. predicted plots of EE (%) and Ld (%) responses after performing all experiments and running the Standard Least Square model for HK-cHy-NPs with emphasis on effective screening. The upper panel shows the responses for each NP preparation presented as EE (%). The lower panel shows the responses for each NP preparation presented as EE (%). In each response plot, the black dots represent the response values obtained after performing the experiments, the horizontal line represents the null hypothesis and the slanted line represents an alternative hypothesis. The shaded area around the slanted line represents the 95% confidence region. RMSE shows the average measured values of difference between the predicted and actual values. RSq (R²) values under each response plot shows how close the data fitted to the regression line (alternative hypothesis).

FIG. 12 shows contour profiler graphs showing effect of various factors on the responses during the synthesis of (a) FL-cHy-NPs. In each figure contour graphs showing responses against factors ‘i’ PCDA vs PPS mPEG, ‘ii’ PCDA vs Drug (FL), and ‘iii’ PPS-mPEG vs Drug (FL). Table ‘iv’ shows set values, predicted values, low (Lo) limit and high (Hi) limit of each response fixed during the preparation of the plots. The shaded portions of each plots showing the responses beyond the fixed Lo or Hi limits. The horizontal and vertical black lines showing the optimized conditions of the respective factor where they cross ‘y’ and ‘x’ axis, respectively.

FIG. 13 shows contour profiler graphs showing effect of various factors on the responses during the synthesis of HK-cHy-NPs. In each figure contour graphs showing responses against factors ‘i’ PCDA vs PPS mPEG, ‘ii’ PCDA vs Drug (HK), and ‘iii’ PPS-mPEG vs Drug (HK). Table ‘iv’ shows set values, predicted values, low (Lo) limit and high (Hi) limit of each response fixed during the preparation of the plots. The shaded portions of each plots showing the responses beyond the fixed Lo or Hi limits. The horizontal and vertical black lines showing the optimized conditions of the respective factor where they cross ‘y’ and ‘x’ axis, respectively.

FIG. 14A shows prediction profiler graphs for optimized synthesis conditions for FL-cHy-NPs as calculated by the quadradic model using the central composite design (CCD) by JMP® pro software. The first value for each parameter on the left axis (particle size, PDI, EE(%), and Ld(%) represents the most optimal condition and predicted results. The second and third values for each parameter show the range of the response values at the same manufacturing conditions. (a) The graph shows conditions for optimal synthesis of. (b) The graph shows conditions for optimal synthesis of HK-cHy-NPs.

FIG. 14B shows prediction profiler graphs for optimized synthesis conditions for HK-cHy-NPs as calculated by the quadradic model using the CCD by JMP® pro software. The first value for each parameter on the left axis (particle size, PDI, EE(%), and Ld(%) represents the most optimal condition and predicted results. The second and third values for each parameter show the range of the response values at the same manufacturing conditions.

FIG. 15 shows size distribution graphs of (i) FL-cHy-NPs and (ii) HK-cHy-NPs showing the average diameter of the NPs and the polydispersity index (PDI) of each type. The inset figures show the visual appearance of the colloidal solution of the same NPs before (1) and after (2) cross-linking.

FIG. 16 shows TEM images of developed hybrid NPs confirming their spherical shape and intact integrity (i) TEM Image of the FL-cHy-NPs (scale 500 nm); right: enlarged NP. (ii) TEM Image of the HK-cHy-NPs (scale 500 nm); right: enlarged NP.

FIG. 17 shows the stability of synthesized FL-cHy-NPs and HK-cHy-NPs in the colloidal solution stored at 4°: (i) the average size and (ii) PDI values of the NPs after 0, 7, 21, 36 and 60 days of storage. The means of each sample were compared using one-way ANOVA and using Dunnett multiple comparison post hoc test with a family-wise alpha threshold confidence level of 0.05 (95% confidence interval). Here, all the values show no difference (ns; p>0.05) from each other suggesting the stability of the NPs at different time points.

FIG. 18 shows the effect of various cryoprotectants on the size (upper panel) and PDI (lower panel) of FL-cHy-NP formulations after lyophilization. The data were analyzed using two-way ANOVA and the group sample means were compared with the blank sample (non-lyophilized) by Dunnett's multiple comparison post-hoc test. The family-wise alpha threshold confidence level was adjusted to 0.05 (95% confidence interval) during the analysis. Here, all the values are showing no difference (p=ns>0.05) with each other suggesting the optimal concentration of the respective cryoprotectant to stabilize the NP preparation (size and PDI).

FIG. 19 shows the effect of various cryoprotectants on the size (upper panel) and PDI (lower panel) of HK-cHy-NP formulations after lyophilization. The data were analyzed using two-way ANOVA and the group sample means were compared with the blank sample (non-lyophilized) by Dunnett's multiple comparison post-hoc test. The family-wise alpha threshold confidence level was adjusted to 0.05 (95% confidence interval) during the analysis. Here, all the values are showing no difference (p=ns>0.05) with each other suggesting the optimal concentration of the respective cryoprotectant to stabilize the NP preparation (size and PDI).

FIG. 20 shows results of cellular internalization of the synthesized cHy-NPs. (a) The fluorescence intensity graph of cell internalization of C6-cHy-NPs in presence of various pathway inhibitors (CPZ, MβCD, AML, and GNT) in comparison with untreated cells. The CPZ showed significant inhibition of fluorescence intensity whereas, no significant difference in fluorescence intensities is showing up in MβCD, AML, and GNT treated groups. (b) The graph shows the effects of various concentrations of pathway inhibitors on cellular internalization of NPs. No significant effect of MβCD, AML, and GNT was shown at the concentration from 2.5-75 μg/mL. (c) The graph shows time-dependent cellular internalization of C6-cHy-NPs at different time intervals (0.25, 1, 2, 3, 4, 5, 6, 8, 10, 24, and 48 h).

FIG. 21 shows fluorescence microscopy images of HEI-OC1 cells (scale 100 μm) after incubation with C6-cHyNPs at different time intervals (i) 0.5 h, (ii) 1 h, (iii) 2 h, (iv) 3 h, (v) 4 h and (vi) 5 h). Green fluorescence was obtained using a FITC filter. The blue stain shows the nuclei of the cells stained by HOECHST-33342 (using DAPI filter). Data in the graphs were compared using two-way ANOVA and each group was compared with the control group “C” by applying Sidik multiple comparisons post-hoc tests. The family-wise alpha threshold confidence level was adjusted to 0.05 (95% confidence interval) during the analysis. (Here P≥0.0=ns, not significant)].

FIG. 22 shows in (a) cell growth of various treatment groups determined using the MTT assay (a). The treatment group of those treated with the combination of FL-cHy-NPs and HK-cHy-NPs did not show a significant reduction in cell growth compared to the control (P>0.05, ns). Also, the combined formulation protects the cells from both 50 and 100 μM concentrations of CisPt. Therefore, the treatment of cells with the combination of FL-cHy-NPs and HK-cHy-NPs has significant protection efficacy against CisPt-induced cell death compared to FL-cHy-NPs or HK-cHy-NPs alone. FIG. 22 shows in (b) intracellular ROS generation assay showing significant ROS generation in the CisPt only treated cells. Significantly low ROS generation have shown in the cells those were treated with FL-cHy-NPs, HK-cHy-NPs and STS.

FIG. 23 shows in (c) fluorescence intensity of MitoSOX reagent corresponding to mitochondrial superoxide generation. The assay indicates significantly low superoxide generation in FL-cHy-NPs and FL/HK-cHy-NPs treatment groups. FIG. 23 shows in (d) caspase 3/7 activation in different treatment groups. The caspase 3/7 activation was significantly lowered in the cells those were treated with FL-cHy-NPs, HK-cHy-NPs and STS. The data were/compared using two-way ANOVA and each group was compared with the control group “C” by applying Dunnett's multiple comparisons post-hoc tests. The family-wise alpha threshold confidence level was adjusted to 0.05 (95% confidence interval) during the analysis. (Here data are presented as Mean±SD; Asterisk; ****, P≤0.0001; ***, P=0.0009; **, P=0.0031; *, P=0.0109).

FIG. 24 is a western blot analysis of the samples after respective treatment with FL-cHy-NPs and HK-cHy-NPs alone or in combination. The blots show proteins associated with apoptotic caspase-3 pathway.

FIG. 25 includes fluorescence microscopy images (scale bar 100 μm) which show the effect of developed FL-cHy-NPs and HK-cHy-NPs individually, and in combination on the CisPt-induced generation of ROS in HEI-OC1 cells. The first column shows the cell nuclei stained with HOECHST-33342. The second column shows the fluorescence of DCF (a ROS marker) in the cells. The third column shows the cells under transmittance light. The fourth column shows the overlay of columns 1, 2, and 3. The last column shows a graph of the ratio of normalized intensities of overall cells and the cells those were producing ROS (being stained with DCFH-DA). Each of columns 1-4 shows results of six treatments, i through vi. (i) Blank untreated cells: the cells those were not treated with any of the NP preparation or CisPt did not show significant ROS generation. The ratio of normalized intensities of total cells and DCF-stained cells was 1.71±0.08. (ii) CisPt treated cells: The cells those were treated with CisPt-only showed significant ROS generation. The ratio of normalized intensities of total cells and DCF-stained cells was 0.99±0.21. (iii) FL-cHy-NPs and CisPt treated cells: the cells treated with FL-cHy-NPs and CisPt did not show significant ROS generation. The ratio of normalized intensities of total cells and DCF-stained cells was 1.71±0.04. (iv) HK-cHy-NPs and CisPt treated cells: the cells treated with HK-cHy-NPs and CisPt did not show significant ROS generation. The ratio of normalized intensities of total cells and DCF-stained cells was 1.63±0.35. (v) FL-cHy-NPs, HK-cHy-NPs and CisPt treated cells: the cells treated with FL-cHy-NPs, HK-cHy-NPs and CisPt did not show significant ROS generation. The ratio of normalized intensities of total cells and DCF-stained cells was 1.66±0.29. (vi) STS and CisPt treated cells: the cells treated with STS and CisPt did not show significant ROS generation. The ratio of normalized intensities of total cells and DCF-stained cells was 1.53±0.23.

FIG. 26 includes fluorescence microscopy images (scale bar 50 μm) which show the effect of developed FL-cHy-NPs and HK-cHy-NPs individually, and in combination on the CisPt-induced HEI-OC1 cells cytotoxicity. The first column shows cell nuclei stained with HOECHST-33342. The second column shows the fluorescence of PI (a dead cell marker) in the cells. The third column shows the cells under transmittance light. The fourth column shows the overlay of columns 1, 2, and 3. The last column shows a graph of the ratio of normalized intensities of overall cells and the cells those were dead (getting PI stain). Each of columns 1-4 shows results of six treatments, i through vi. (i) Blank untreated cells: the cells those were not treated with any of the NP preparation or CisPt did not show significant cell death. The ratio of normalized intensities of total cells and PI-stained cells was 2.62±0.05. (ii) CisPt treated cells: The cells those were treated with CisPt-only showed significant cell death. The ratio of normalized intensities of total cells and PI-stained cells was 1.06±0.08. (iii) FL-cHy-NPs and CisPt treated cells: the cells treated with FL-cHy-NPs and CisPt did not show significant cell death. The ratio of normalized intensities of total cells and PI-stained cells was 1.87±0.22. (iv) HK-cHy-NPs and CisPt treated cells: the cells treated with HK-cHy-NPs and CisPt did not show significant cell death. The ratio of normalized intensities of total cells and PI-stained cells was 1.97±0.15. (v) FL-cHy-NPs, HK-cHy-NPs and CisPt treated cells: the cells treated with FL-cHy-NPs, HK-cHy-NPs and CisPt did not show significant cell death. The ratio of normalized intensities of total cells and PI-stained cells was 2.41±0.0.32. (vi) STS and CisPt treated cells: the cells treated with STS and CisPt did not show significant cell death. The ratio of normalized intensities of total cells and PI-stained cells was 2.03±0.02. [The microscopy data were compared using one-way ANOVA and each group was compared with the control group (c; CisPt only treated) by applying Dunnett's multiple comparisons posthoc test. The family-wise alpha threshold confidence level was adjusted to 0.05 (95% confidence interval) during the analysis and p<0.05 was considered as a significantly different group].

FIG. 27 shows SEM analysis of hydrogel after lyophilization (scale 100 μm); SEM images of FL-cHy-NPs kept at 25° C. (i), and 37° C. (ii); SEM images of HK-cHy-NPs kept at 25° C. (iii), and 37° C. (iv).

FIG. 28 shows FE-SEM images analysis of nanoparticles-embedded-in-hydrogel formulation by deep learning (scale 2.5 μm); Segmentation of FL-cHy-NPs at 25° C. (i) and 37° C. (ii); Segmentation of HK-cHy-NPs at 25° C. (iii) and 37° C. (iv). In each of these segmentations blue regions represent deep learning segmentation model identified as particles.

FIG. 29 shows results of an in vitro drug release study of FL (upper panel) and HK (lower panel) at 25° C. and 37° C. for up to 1 month.

FIG. 30 uses the data of FIG. 29 to fit cumulative percent drug release (CPDR) of FL and HK release with the Korsmeyer-Peppas Model from the respective hydrogel formulation at 25 and 37° C.

FIG. 31 shows that FL and HK protects from cisplatin ototoxicity in zebrafish. Images A-L are representative micrographs of neuromast hair cells immunostained red for the hair cell marker otoferlin. D-F: Fl alone at 33 mM, 17 mM or 2 mM concentrations. G-I: HK alone at 33 mM, 17 mM or 2 mM concentrations. J-L: FL and HK in combination at 17 mM, 8.5 mM or 3 mM concentrations (each). NP=empty nanoparticles. Scale bar: 10 mm.

FIG. 32 shows quantification of the number of hair cells per neuromast in the results of FIG. 31. Results are expressed as percentage of protection, with 100% representing control animals and 0% cisplatin-treated fish. A maximum of three neuromast were inspected per fish were inspected in 10-12 fish. Statistical analysis: One-way ANOVA followed by Dunnett post-test for multiple comparisons. ***P<0.01 compared to cisplatin alone.

### ABBREVIATIONS

ACN: acetonitrile,
AI: Artificial intelligence,
AML: Amiloride,
ANOVA: analysis of variance,
BUC: bucillamine,
BUC-NC: Bucillamine nanocarrier,
BUC-PPS-mPEG₂₀₀₀-NC: Bucillamine-Polypropylene sulfide-methyl polyethyleneglycol-2000 nanocarrier,
° C.: degrees Celsius,
cHy-NP: Crosslinked hybrid nanoparticle,
CCD: Central composite design,
CisPt: Cisplatin: cis-Diammineplatinum(II) Dichloride,
CIHL: Cisplatin-induced hearing loss,
CIO: Cisplatin-induced ototoxicity,
CPZ: Chlorpromazine,
C-6: Coumarin-6,
C940: Carbomer 940,
DCFH-DA: Dichloro-dihydro-fluorescein diactate,
DCM: dichloromethane,
DI: Deionized,
DL: Detection limit,
DLS: Dynamic light scattering,
DLT: diltiazem,
DLT-NC: Diltiazem nanocarrier,
DLT-PPS-mPEG₂₀₀₀-NC: Diltiazem-Polypropylene sulfide-methyl polyethyleneglycol-2000 nanocarrier,
DoE: Design of experiment,
dpf: day postfertilization,
EE: Encapsulation efficiency,
FE-SEM: Field emission scanning electron microscopy,
FL: Flunarizine,
FL-cHy-NP: Flunarizine-crosslinked hybrid nanoparticle,
FLICA: Fluorescent-labeled inhibitors of caspases,
GNT: Genistein,
hr: hour,
HCl: hydrochloric acid,
HEI-OC1: House Ear Institute-Organ of Corti 1 cells,
HK: Honokiol,
HK-cHy-NP: Honokiol-crosslinked hybrid nanoparticle,
HL: Hearing loss,
HPLC: High-performance liquid chromatography,
HY: hydrogel,
IHC: inner ear hair cell,
kHz: kilohertz,
Korsmeyer-Peppas model,
λex/em: excitation/emission wavelengths,
LC-MS: Liquid chromatography-Mass spectroscopy,
Ld: Drug loading,
m: meter,
MβCD: methyl-β-cyclodextrin,
min: minute,
mL: milliliter,
mPEG: polyethylene glycol monomethyl ether,
mPEG₂₀₀₀: polyethylene glycol monomethyl ether-2000,
ms: millisecond,
μg: microgram,
μL: microliter,
am: micrometer,
NC: nanocarrier,
nm: nanometer,
NMR: Nuclear magnetic resonance,
NP: nanoparticle,
NTA: nanoparticle tracking analysis,
OHC: outer hair cell,
PBS: phosphate buffer saline,
PCDA: 10,12-Pentacosadiynoic acid,
PDI: Polydispersity index,
PEG: polyethylene glycol,
PFA: paraformaldehyde,
PI: propidium iodide,
PPS: polypropylene sulfide,
PPS-mPEG: Polypropylene sulfide-methyl ether polyethyleneglycol,
PPS-mPEG₂₀₀₀: Polypropylene sulfide-methyl ether polyethyleneglycol-2000,
P188: Poloxamer 188,
P407: Poloxamer 407,
RMSE: Root Mean Squared Error,
rpm: revolutions per minute,
ROS: Reactive oxygen species,
SDS-PAGE: Sodium dodecyl sulfate-polyacrylamide gel electrophoresis,
sec: second,
SEM: Scanning Electron Microscopy,
SI: Scatter index,
SIRT-3: sirtuin 3,
STS: Sodium thiosulphate,
TBS-T: Tris-buffered saline with Tween 20,
TEA: triethylamine,
TEM: Transmission electron microscopy,
TIHL: Treatment-induced hearing loss,
UV-vis: ultraviolet-visible,
w/v: weight/volume,
w/w: weight/weight,
%: percent.

### DETAILED DESCRIPTION

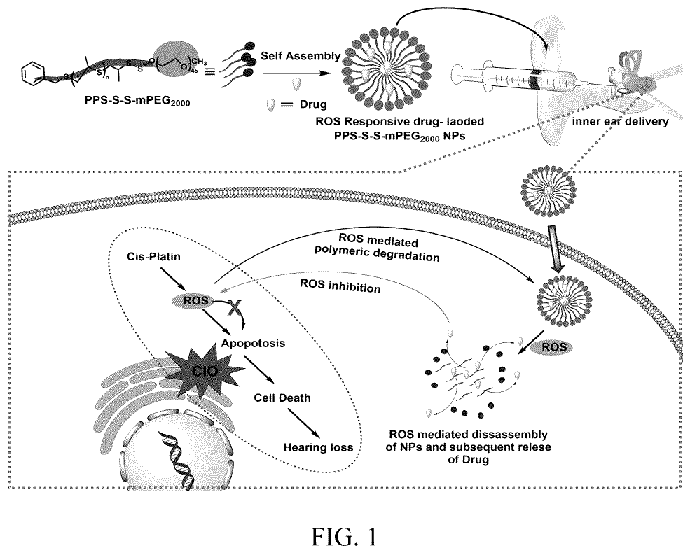

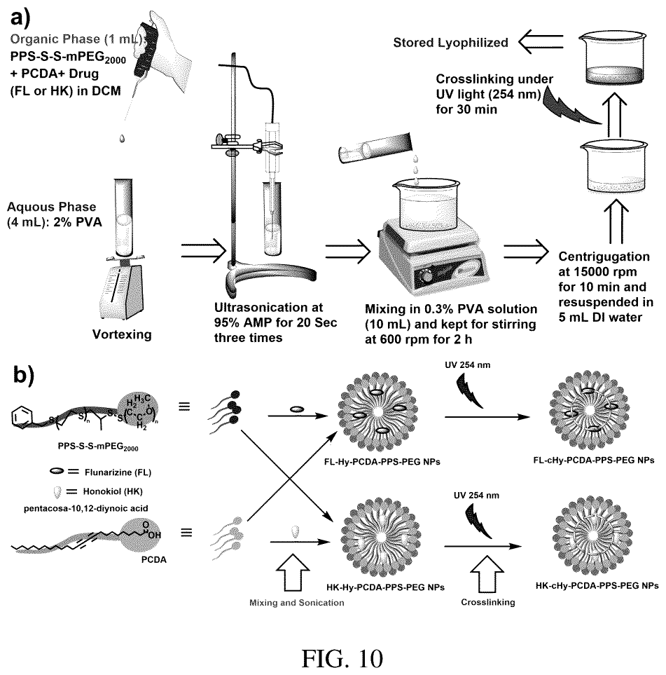

The present disclosure discloses prophylactic treatments for prophylactic prevention, mitigation, or reduction of drug-induced ototoxicity and associated hearing loss by providing an effective drug delivery system which releases a therapeutic drug upon exposure to ROS. In at least one non-limiting embodiment, the present disclosure is directed to a composition comprising a thermoresponsive hydrogel formulation comprising therapeutic drug-loaded crosslinked hybrid nanoparticles in a blend of a poloxamer and a carboxamer (e.g., P407, P188, and C940) hydrogel which provides sustained drug release and release kinetics, for example at 25° C. and 37° C. The drug-induced ototoxicity or drug-induced hearing loss may be induced by a platinum-based drug selected from cisplatin, carboplatin, oxaliplatin, nedaplatin, heptaplatin, lobaplatin, miriplatin, tetraplatin, iproplatin, satraplatin, ormaplatin, and oxoplatin. The mechanism is schematically represented in FIGS. 1 and 10, for example.

### Example 1

### Methods

Novel dual stimuli-responsive nanoformulations have been investigated for the synthesis of ROS-responsive polymers, and thermo-responsive polymers and their characterization using ¹H NMR. Drug loaded NCs comprising BUC and/or DLT in micelles of PPS-mPEG₂₀₀₀ were prepared by nanoprecipitation methods. The BUC-PPS-mPEG₂₀₀₀-NCs and DLT-PPS-mPEG₂₀₀₀-NCs were characterized using DLS and NTA and TEM images. The EE % and DL % of NCs were determined using HPLC and Ellman's assays. The long-term storage stability over two months was checked by using DLS for size and PDI. In vitro assessments were performed on the HEIOC1 cell lines for MTT and Live-dead cell assays for biocompatibility and cellular uptake analysis, respectively. The cytoprotective effect of BUC-NCs, DLT-NCs and BUC/DLT-PPS-mPEG₂₀₀₀-NCs, were studied after treating with various regimens and different concentration with and without cisplatin. The ROS-Scavenging effect nanoformulation was confirmed by the DCFH-DA assay, and FLICA assays. The final formulation, named NanoSensoGel, comprising a combination of a thermo-responsive P407 hydrogel, and the ROS-responsive BUC/DLT loaded PPS-mPEG₂₀₀₀-NCs, was characterized for its bio-responsiveness, a novel method was established to perform the in vitro drug release, and release kinetics mechanism were studied for dual drug release.

### Results

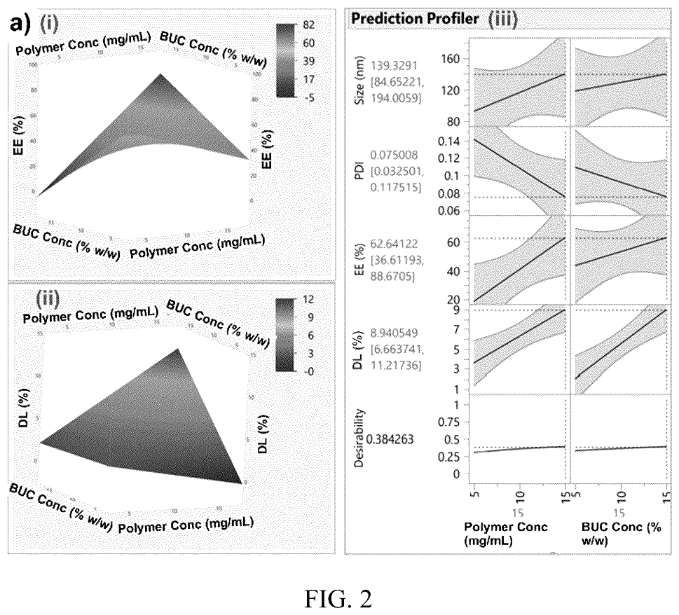

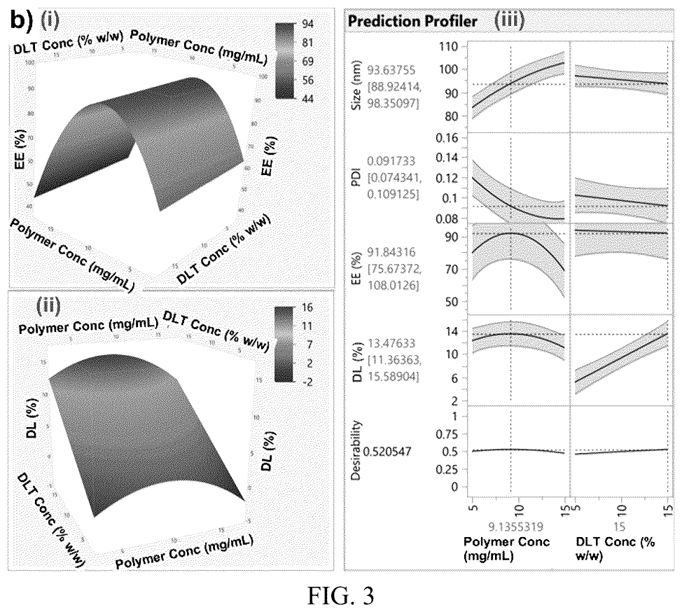

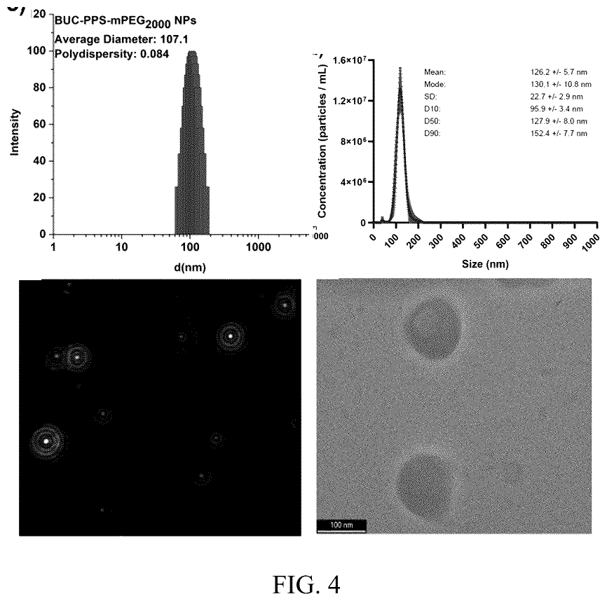

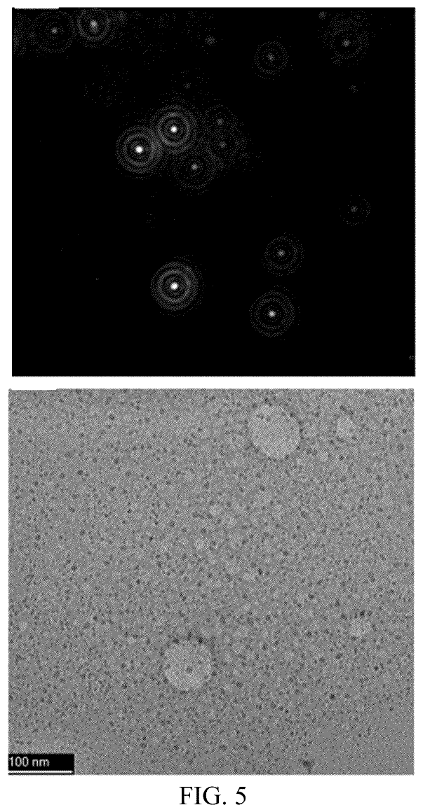

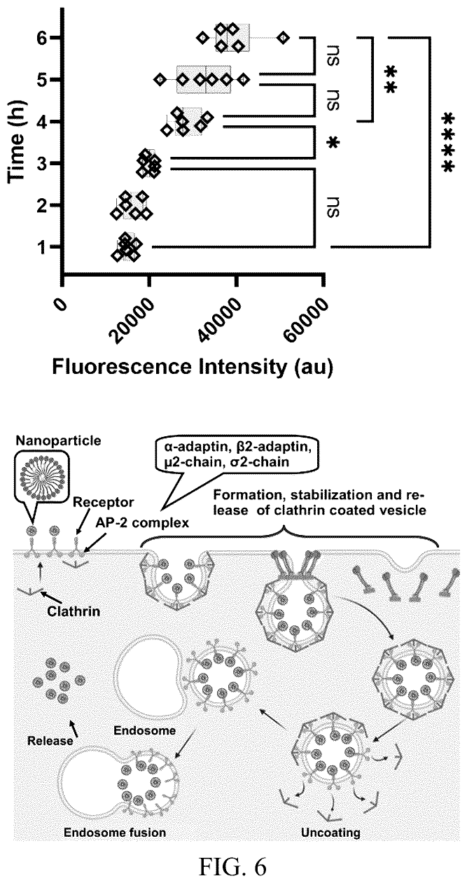

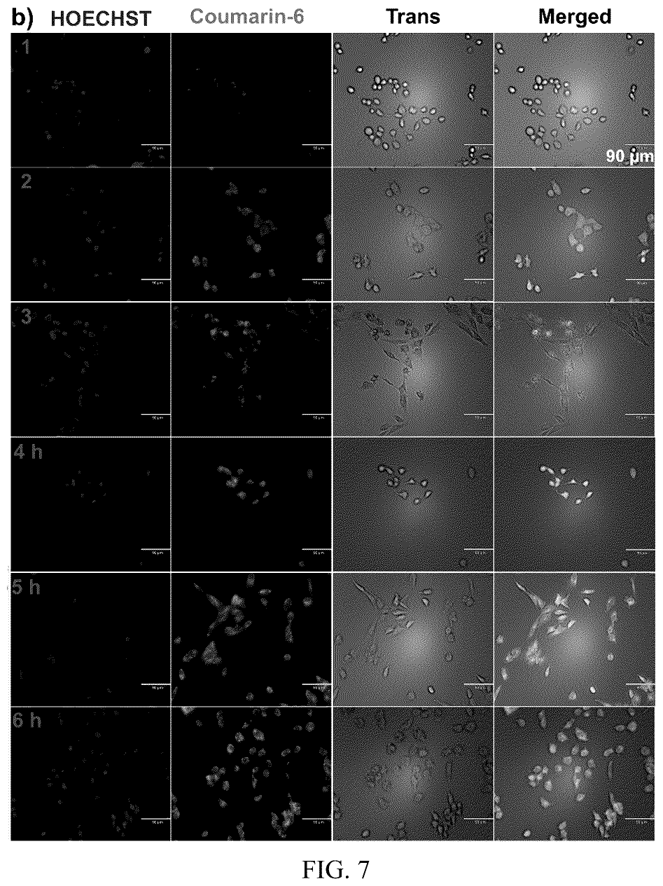

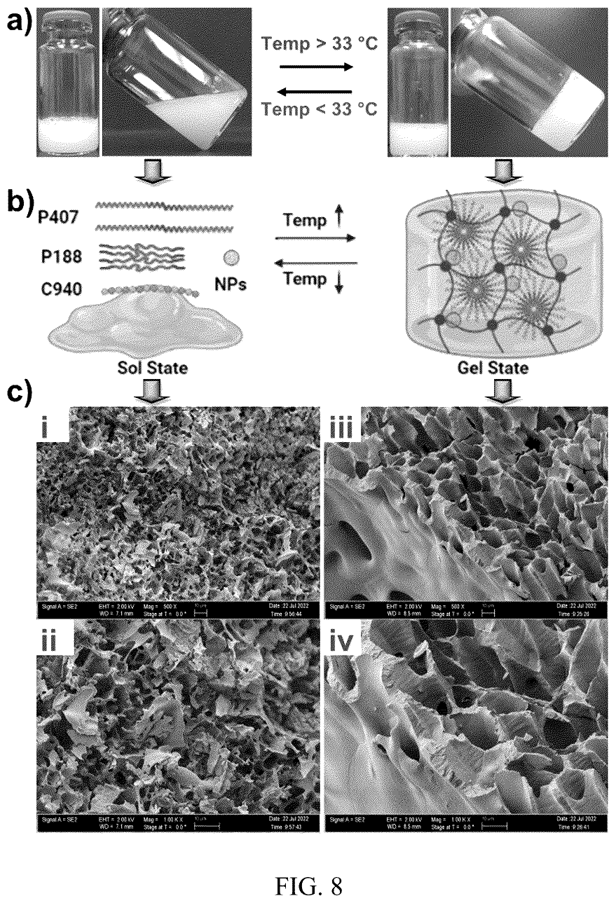

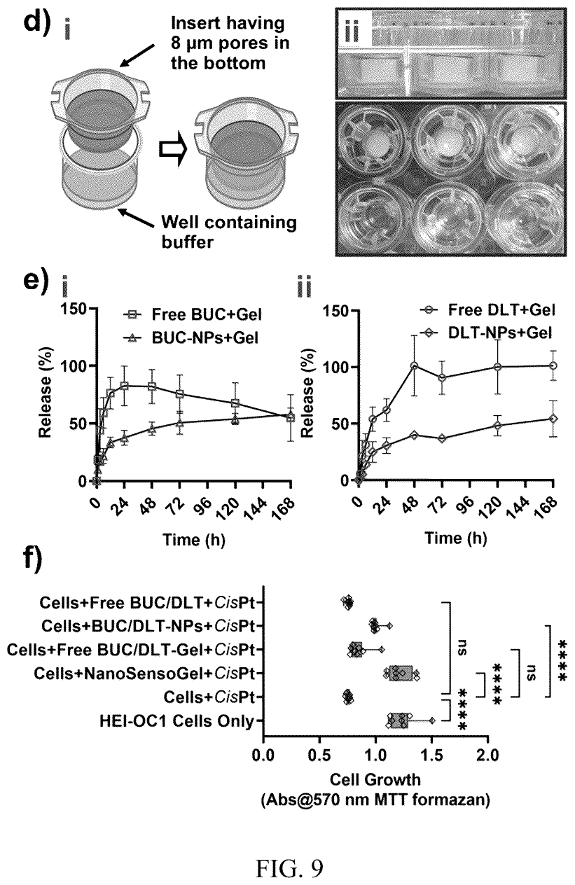

NMR spectra showed the designated proton peak of the ROS-responsive and thermos-responsive polymers successfully synthesized using multistep. The average sizes of BUC-PPS-mPEG₂₀₀₀-NCs, and DLT-PPS-mPEG₂₀₀₀-NCs, were found ˜107.1 nm and 98.8 nm, respectively and PDI<0.3 confirmed that most of the NCs have the same size. The NC preparations appeared spherical and, the D10/D50/D90 values of BUC-NCs, and DLT-NCs were found to be 95.9/127.9/154.4 nm and 84.3/115.8/157.4 nm, respectively identified by NTA. The morphological analysis of developed NCs confirmed the spherical shape and the size of ˜100 nm using NTA and TEM (FIGS. 2-5). The EE/DL of the BUC and DLT in the respective NCs were found to be 56±4/8.38% and 87±7/12.30%. BUC-PPS-mPEG₂₀₀₀-NCs alone or in combination with DLT-PPS-mPEG₂₀₀₀-NCs have demonstrated as highly efficient as ROS scavengers in HEI-OC1 cells and could be beneficial to protect CIO (FIGS. 6-7). BUC-PPS-mPEG₂₀₀₀-NCs showed efficient antioxidant activity confirmedly ABTS⁺ radical scavenging assay. Both BUC- and DLT-loaded PPS-mPEG₂₀₀₀-NCs were found to be highly biocompatible with HEI-OC1 cells, therefore their cytoprotective effect after CisPt exposure was determined. The results were further confirmed by fluorescence microscopy which confirmed that the BUC is highly responsible for the inhibition of the caspase 3/7 activation in the CisPt-exposed HEI-OC1 cells. Caspase inhibition by developed BUC-entrapped NPs could be highly desirable for reduced CisPt-induced cytotoxicity and CIO, subsequently. Several characterizations of NanoSensoGel formulation are shown in FIGS. 8-9 including sol-gel state mechanism. The sample observed under SEM showed a well-arranged honeycomb-type pattern in the structure (scale 10 μm). The release mechanism fitted well with the KP-model. It was observed the ‘n’ values of BUC and DLT release from Free BUC/DLT-Gel and NanoSenosGel formulations were found to be between 0.45 to 1 (0.45<n<1) which suggests the non-Fickian transport.

The disclosed cytoprotective NanoSensoGel formulation was successfully developed as a preventative therapeutic agent for the treatment of the CIO and associated HL. This is the first investigation engineered with dual-stimuli and dual-drug combination against irreversible IHC loss. Moreover, this technology can be utilized for other inner ear diseases such as Age-related HL and Noise-induced HL.

### Example 2

### Methods

Flunarizine (FL, TCI), cis-Diammineplatinum(II) Dichloride (CisPt, TCI), Chlorpromazine (CPZ, TCI), Amiloride (AML, TCI), methyl-β-cyclodextrin (MβCD, TCI), Genistein (GNT, TCI), triethylamine (TEA, Fischer Chemical), acetonitrile (ACN, Fischer Chemical), Methanol (Fischer Chemical), dichloromethane (DCM, Fischer Chemical), propidium iodide (PI, MP Biomedicals) Coumarin-6 (C6, Thermo Fischer), 10,12-Pentacosadiynoic acid (PCDA; TCI), HOECHST 33342 (Tocris Biosciences), 2′,7′-Dichlorofluorescein 3′,6′-diacetate (DCFH₂-DA, Thermo Fischer), Poloxamer 188 (P188, Alfa Aesar), hydrochloric acid (HCl, Fischer Chemicals) and Carbomer 940 (C940, Spectrum) were purchased from Fischer Scientific, USA. Poloxamer 407 (P407) and Trypsin-EDTA solution (0.25%) were purchased from Millipore Sigma, USA. Polypropylene sulfide-methyl ether polyethyleneglycol (PPS-mPEG) was synthesized inhouse. Honokiol (HK, >98%) was purchased from MCS Formulas B. V., Netherlands. The cell culture media, reagents, and PBS were procured from Millipore Sigma, USA. MitoSOX™ Cat: M36008, assay kit was received as a kind gift from Invitrogen, USA.

HPLC method development of Flunarizine (FL) and Honokiol (HK)

The qualitative and quantitative analytical methods of the FL and HK were developed on an HPLC system utilizing a previous protocol with some modifications [33,34]. The HPLC system (1260 Infinity, Agilent Technologies, Santa Clara, CA, USA) equipped with VWD (UV-vis detector) was utilized for the method development. Different concentrations of FL and HK (0.048828-100 μg/mL) were prepared from the stock (2 mM) in methanol. The samples were run in HPLC according to the International Conference on Harmonization (ICH) protocol for qualitative and quantitative method validation (3 samples of each concentration per day for three consecutive days) utilizing the method parameters listed in Table 1. Validation calculations were performed using the smallest five concentrations (0.048828-0.78125 μg/mL (Tables 2-3). The standard curves for each data set were drawn and equations were determined. The limit of detection (LOD) and limit of quantification (LOQ) were further determined using the slopes of the standard equations by following formulas [35]:

$$LOD = \frac{3.3 σ}{S} \qquad ( Eq . 1 )$$

$$LOQ = \frac{10 σ}{S} \qquad ( Eq . 2 )$$

where, σ is the standard deviation of the slope, and S is the slope (mean) of the graph.

TABLE 1
HPLC method parameters for FL and HK qualitative and quantitative analysis

| Method parameters | FL | | HK | |
|---|---|---|---|---|
| Mobile Phase (MP) | A: 0.1% TEA pH 3.5 | | A: ACN:Water (5:4) | |
| | B: Methanol (100%) | | B: Methanol (100%) | |
| | Ratio of A:B = 25:75 | | Ratio of A:B = 45:55 | |
| Column | C-18 Phenomenex Columbus | | C-18 Phenomenex Columbus | |
| | 5 μ, 100 × 4.8 mm, 25° C. | | 5 μ, 100 × 4.8 mm, 30° C. | |
| Injection volume | 10 | μL | 10 | μL |
| Flow Rate | 1 | mL/min | 1 | mL/min |
| Wavelength | 252 | nm | 294 | nm |
| Acquisition time | 4 | min | 4 | min |

TABLE 2
HPLC validation table for the determination of LOD and LOQ of FL.

| | Symbol | FL1 | FL2 | FL3 | Avg | SD |
|---|---|---|---|---|---|---|
| Slope | S | 33.9095 | 35.51151 | 35.2702 | 34.89705 | 0.863756 |
| Intercept | b | 1.12778 | 1.702778 | 1.82917 | 1.553241 | 0.373842 |
| Number of tests | N | 3 | 3 | 3 | | |
| SE of Intercept | | 0.51769 | 0.378231 | 0.36947 | 0.421796 | 0.083164 |
| SE of Regression | σ | 0.76468 | 0.558685 | 0.54574 | 0.623034 | 0.122842 |
| LOD (μg/mL) | $LOD = \frac{3.3 σ}{S}$ | 0.07442 | 0.051917 | 0.05106 | 0.059132 | 0.013244 |
| LOQ (μg/mL) | $LOD = \frac{10 σ}{S}$ | 0.22551 | 0.157325 | 0.15473 | 0.179187 | 0.040135 |

TABLE 3
HPLC validation table for the determination of LOD and LOQ of HK.

| | Symbol | H1 | H2 | H3 | Average | SD |
|---|---|---|---|---|---|---|
| Slope | S | 33.642 | 31.09 | 36.789 | 33.84033 | 2.854672 |
| Intercept | b | 1.6153 | 1.3681 | 1.6028 | 1.528733 | 0.139253 |
| Number of tests | N | 3 | 3 | 3 | | |
| SE of Intercept | | 0.23418 | 0.263024 | 0.298073 | 0.265092 | 0.031997 |
| SE of Regression | σ | 0.345906 | 0.388512 | 0.440283 | 0.391567 | 0.047263 |
| LOD (μg/mL) | $LOD = \frac{3.3 σ}{S}$ | 0.033931 | 0.041238 | 0.039494 | 0.038221 | 0.003816 |
| LOQ (μg/mL) | $LOD = \frac{10 σ}{S}$ | 0.10282 | 0.124964 | 0.119678 | 0.115821 | 0.011565 |

### Preparation of FL-cHy-NPs and HK-cHy-NPs

The FL-cHy-NPs and HK-cHy-NPs were synthesized using the ultrasonic nanoprecipitation solvent evaporation method as depicted in FIG. 10(a). During the synthesis of the NPs, the hydrophobic moieties of the PPS-mPEG and PCDA molecules come together and form the core of the NPs while the hydrophilic moieties (PEG and —COOH) arranged on the surface of the nanoparticles in contact with the aqueous media. The FL and HK drugs are generally entrapped in the core of the NPs during the synthesis due to hydrophobic nature of drugs. The color change from pale white to dark blue was observed after the UV irradiation (254 nm) for 30 min confirming the crosslinking of the PCDA molecules (FIG. 10(b)). The cross-linking of PCDA molecules stabilizes the NP preparation in the solution and help them to retain their morphological characteristic during storage and further processing (lyophilization, saline reconstitution, and entrapping in hydrogels, creams, capsules, etc.) of the formulation.

### Preparation of FL-cHy-PCDA-PPS-mPEG-NPs (FL-cHy-NPs) and HK-cHy-PCDA-PPS-mPEG-NPs (HK-cHy-NPs)

For the final synthesis of FL-cHy-NPs and HK-cHy-NPs, briefly, PPS-mPEG₂₀₀₀ (14 and 15 mg for FL and HK, respectively), PCDA (50% w/w of PPS-mPEG₂₀₀₀) and FL or HK (12.5% w/w of PPS-mPEG₂₀₀₀ and PCDA) were dissolved in 1 mL of in a microcentrifuge tube. Any suitable organic solvent may be used in place of DCM. In certain non-limiting embodiments of the present disclosure, the PPS-mPEG is dissolved in an organic solvent (e.g., DCM) in a range of about 12 mg PPS-mPEG per ml of solvent to about 17 mg PPS-mPEG per ml solvent. In a 15 mL centrifuge tube containing 2% PVA solution (4 mL), this mixture was mixed in under vortexing and then ultra-sonicated using a 20 kHz ultrasonicator (Fischer Scientific, USA) for 20 sec (three times). The colloidal emulsion was then transferred to a small beaker containing 10 mL of 0.3% PVA solution and kept for stirring at 600 rpm for 2 h. The colloidal solution of NPs then centrifuged at 15000 rpm for 10 min and after discarding the supernatant, the pellet was resuspended in 5 mL DI water. The suspended NP solution was kept under UV light (254 nm) for cross-linking. Before the final synthesis of NPs, various synthesis parameters were optimized by design of experiment (DoE) approach using central composite design (CCD) (JMP® Pro 16, SAS, NC, USA) to obtain favorable size, PDI, encapsulation efficiency, EE(%), and loading efficiency, Ld(%) [36].

In JMP® pro 16 software, the response surface design window was opened by clicking on “DOE”>“Classical”>“Response Surface Design”. Four responses, (i) Particle Size (nm), (ii) PDI, (iii) EE(%), and (iv) Ld(%), were added in the window. The goal of the particle size and PDI responses were kept “minimize” while the goal for EE(%) and Ld(%) was kept “maximize”. The upper and lower limits were also setted up in the table. In the factors section, three continuous factors were added as (i) PPS-mPEG (mg/mL), (ii) PCDA (% w/w of PPS-mPEG), and (iii) drug (FL or HK, % w/w of PPS-mPEG and PCDA). The values (−1 and +1) for PPS-mPEG (5 and 20), PCDA (10 and 100), and drugs (5 and 20) were added. After adding all responses and factors clicked on ‘continue’ button and central composite design was selected and clicked on ‘continue’. The “display and modify design” section then appeared where the axial value was kept ‘1.000’, ‘On Face’ was selected and the run order selected ‘Keep the same’ and then clicked on ‘Make Table’. The CCD design table was generated and saved. The quantities of factors for each run were calculated carefully and final organic phase (1 mL) containing all three factors was obtained for each run. The synthesis of NPs was done as described in the previous section.

The Particle size and PDI value of NPs in each sample were determined by DLS. The EE(%) and Ld(%) of drugs in each sample was determined using following formulas:

$$EE ( \% ) = \frac{C}{{C}_{0}} \times 100 \qquad ( Eq . 3 )$$

$$DL ( \% ) = \frac{C}{{P}_{0}} \times 100 \qquad ( Eq . 4 )$$

where, C is the concentration of the drug into the nanoparticles, C₀ is the initial drug concentration, P₀ is the concentration of added polymer.

All acquired data was inputted into the CCD table (Tables 4-5), then clicked on the ‘Model’ to open fit model window. In the ‘Pick Role Variable’ section, all four responses added to ‘Y’ section. The personality was settled as ‘Standard Least Square’ and emphasis was set as ‘Effect Screening’, then the model was run by hitting ‘run’ button. In the least square fit section, the non-significant factors were removed from the ‘Effect Summary’ table one by one. The ANOVA, Lack of Fit, Actual by Predicted Plot, Residual by Predicted Plot, and Studentized Residuals for each response were obtained from least square fit model to evaluate if the model is fitting well. The SI % of each response was determined using RMSE and mean response (X̅) by applying the following formula:

$$SI ( \% ) = \frac{R M S E}{\overline{X}} \times 100 \qquad ( Eq . 5 )$$

TABLE 4
The DoE-CCD table for the synthesis optimization of FL-cHy-PCDA-PPS-NPs

| | | PPS-mPEG | DA | FL (w/w % of | Particle | | | |
|---|---|---|---|---|---|---|---|---|
| Run | Pattern | (mg/mL) | (w/w %) | PPS and DA) | Size(nm) | PDI | EE(%) | Ld(%) |
| 1 | −−− | 5 | 10 | 5 | 245.6 | 0.107 | 65.37 | 3.27 |
| 2 | −−+ | 5 | 10 | 20 | 253.5 | 0.193 | 46.25 | 9.25 |
| 3 | −+− | 5 | 100 | 5 | 281.1 | 0.285 | 51.60 | 2.58 |
| 4 | −++ | 5 | 100 | 20 | 301.2 | 0.316 | 31.20 | 6.24 |
| 5 | +−− | 20 | 10 | 5 | 220.1 | 0.121 | 66.81 | 3.34 |
| 6 | +−+ | 20 | 10 | 20 | 241.3 | 0.208 | 40.10 | 8.02 |
| 7 | ++− | 20 | 100 | 5 | 310.6 | 0.143 | 79.35 | 3.97 |
| 8 | +++ | 20 | 100 | 20 | 325.2 | 0.222 | 46.30 | 9.26 |
| 9 | a00 | 5 | 55 | 12.5 | 293.1 | 0.266 | 26.80 | 3.35 |
| 10 | A00 | 20 | 55 | 12.5 | 281.3 | 0.169 | 89.34 | 11.17 |
| 11 | 0a0 | 12.5 | 10 | 12.5 | 215.2 | 0.173 | 63.56 | 7.95 |
| 12 | 0A0 | 12.5 | 100 | 12.5 | 279.7 | 0.232 | 62.13 | 7.77 |
| 13 | 00a | 12.5 | 55 | 5 | 246.8 | 0.158 | 91.37 | 4.57 |
| 14 | 00A | 12.5 | 55 | 20 | 263.2 | 0.238 | 61.37 | 12.27 |
| 15 | 000 | 12.5 | 55 | 12.5 | 245.7 | 0.179 | 83.25 | 10.41 |
| 16 | 000 | 12.5 | 55 | 12.5 | 242.2 | 0.167 | 85.61 | 10.70 |

TABLE 5
The DoE-CCD table for the synthesis optimization of HK-cHy-PCDA-PPS-NPs

| | | PPS-mPEG | PCDA | HK (w/w % of | Particle | | | |
|---|---|---|---|---|---|---|---|---|
| Run | Pattern | (mg/mL) | (w/w %) | PPS and DA) | Size(nm) | PDI | EE(%) | Ld(%) |
| 1 | −−− | 5 | 10 | 5 | 233.4 | 0.197 | 51.33 | 2.567 |
| 2 | −−+ | 5 | 10 | 20 | 245.7 | 0.203 | 31.91 | 6.382 |
| 3 | −+− | 5 | 100 | 5 | 283.3 | 0.305 | 64.1 | 3.205 |
| 4 | −++ | 5 | 100 | 20 | 317.6 | 0.36 | 23.6 | 4.72 |
| 5 | +−− | 20 | 10 | 5 | 211.8 | 0.139 | 63.66 | 3.183 |
| 6 | +−+ | 20 | 10 | 20 | 226.6 | 0.206 | 36.3 | 7.26 |
| 7 | ++− | 20 | 100 | 5 | 301.4 | 0.254 | 91.26 | 4.563 |
| 8 | +++ | 20 | 100 | 20 | 285.1 | 0.287 | 59.36 | 11.87 |
| 9 | a00 | 5 | 55 | 12.5 | 282.2 | 0.258 | 32.89 | 4.111 |
| 10 | A00 | 20 | 55 | 12.5 | 296.5 | 0.209 | 97.4 | 12.18 |
| 11 | 0a0 | 12.5 | 10 | 12.5 | 225.6 | 0.192 | 59.33 | 7.416 |
| 12 | 0A0 | 12.5 | 100 | 12.5 | 287.7 | 0.282 | 85.13 | 10.64 |
| 13 | 00a | 12.5 | 55 | 5 | 241.7 | 0.108 | 88.63 | 4.432 |
| 14 | 00A | 12.5 | 55 | 20 | 251.7 | 0.188 | 42.87 | 8.574 |
| 15 | 000 | 12.5 | 55 | 12.5 | 275.7 | 0.182 | 88.69 | 11.09 |
| 16 | 000 | 12.5 | 55 | 12.5 | 262.3 | 0.159 | 86.37 | 10.8 |

After confirming good fit, the predicted formula column for each response was added to the response table by clicking on ‘red down arrow’ (beside each response)>‘Save Columns’>‘Prediction Formula’. The contour profiler to get the optimized factor for the synthesis of FL-cHy-NPs and HK-cHy-NPs were drawn using the prediction formula. To prepare the contour profiler, clicked on ‘Graph’>‘Contour Profiler’ then the prediction formula for each response was added in ‘Y, prediction formula’ section and clicked ‘OK’. The effect of a combination of two factors at a time was determined to get the desired response. The optimal factor concentrations for a desired response were then calculated using the prediction profiler graphs in the least square fit window.

### Characterization of FL-cHy-NPs and HK-cHy-NPs

The size and PDI of the synthesized NPs were determined using DLS. Samples were prepared by adding 10 μL of freshly prepared NPs in 990 μL of DI water and analyzed using a DLS instrument equipped with a 635 nm laser (Brookhaven Instruments, Holtsville, NY, USA). Further, the morphology and size were confirmed using TEM analysis. Briefly, the diluted colloidal solutions (10 μL) were dropped on the carbon-coated copper grid (200 mesh). After 5 min, the grids were washed using DI water 2 times (30 sec) and 10 μL staining solution (uranyl acetate, 2% w/v) was dropped on the grids. After 1 min, the grids were washed again using deionized water 2 times and air dried for 10 min. The samples were analyzed using TEM (Hitachi H-7600, Hitachi, Japan) at 80 kV and the images were captured using NANOSPRT12 camera with 800 (ms)×4 drift frames exposure at normal contrast.

### Storage Stability and Freeze Drying

The storage stability of the developed FL-cHy-NPs and HK-cHy-NPs was determined by keeping them at 4° C. in the solution for at least 60 days. The size and PDI values for stored samples at 0, 7, 21, 36, and 60 day interval were recorded using DLS. The effect of various cryoprotectants (glucose, mannitol, sucrose, and trehalose) was determined. Momentarily, different amounts (1, 2.5, 5, 7.5, and 10% w/v) of cryoprotectants were added to the colloidal solution of NPs and lyophilized using Triad Benchtop Freeze Dryer (Labconco, Kansas City, MO, USA). Freeze-dried samples were reconstituted in DI water, then size and PDI values of the samples were recorded using DLS.

### Field Emission Scanning Electron Microscopy (FE-SEM) Image Based Analytical Study

Utilizing the high resolution field emission scanning electron microscopy images were analyzed for a quantitative and qualitative comparison. Images of the FL-cHy-NPs and HK-cHy-NPs hydrogels preparations were taken at a range of magnifications from 5 to 35 KX. Using the DigiM I2S software platform the 5KX magnification images, of the FL-cHy-NPs and HK-cHy-NPs hydrogels stored at 25° C. and 37° C., were analyzed and segmented to determine particle size and distribution throughout the visible scene. A combination of AI-based image analytical techniques was used to determine the particle location within the scene, including a deep learning semantic segmentation model. The deep learning model was trained using a supervised learning pattern, in which the inferenced regions of the images were manually corrected and passed back to the model as additional training data. This process was repeated until a segmentation with little to no visible issues remained. After segmentation, the drug particles were analyzed for their morphology, including size, shape and percentage of the image.

### Cellular Internalization Study

First, blank cHy-NPs (cHy-NPs, without FL or HK encapsulation) were prepared as described below. In, 1 mL colloidal solution of cHy-NPs, 1 mg coumarin-6 (C-6) was added and the mixture was stirred at 200 rpm overnight at room temperature. The NP solution was then centrifuged at 2000 rpm for 5 min to remove unloaded C-6. The supernatant containing C-6-cHy-NPs was transferred to a fresh microcentrifuge tube. The HEI-OC1 (House Ear Institute-Organ of Corti 1) cells were seeded in 96 well plate (10000 cells per well) overnight. After washing with fresh growth media, the media (100 μL) containing 40 μg/mL CPZ, MβCD, GNT, or AML was added to separate columns (2 columns kept untreated as a control group). After 1 h of incubation at 33° C. (5% CO₂), 2.5 μL of C6-cHy-NPs (10× diluted with media) were added to each well. The plate was incubated for 4 h and the cells were washed using PBS. After adding fresh PBS, the fluorescence intensity of each well was recorded using a multi-well plate fluorescence spectrophotometer (Synergy 2, BioTek, USA) at λex/em 460/505. In another experiment to confirm the effect of various inhibitors, the cells (20000 per well) were seeded overnight, and incubated with different concentrations (0, 2.5, 5, 10, 20, 30, 40, 50, and 75 μg/mL) of MβCD, GNT, or AML. After treatment, incubation, and washing, the fluorescence intensity of each well was recorded as described above. Furthermore, to study the time of cellular internalization the cells (20000 per well) were treated with C-6-cHy-NPs (5 μL, 10× diluted), and the fluorescence of the samples was recorded after washing at different time intervals (0.25, 0.5, 1, 2, 3, 4, 5, 6, 8, 10, 24, and 48 h) of incubation. For microscopy analysis, the cells were seeded in a 6 well plate (1×10⁵ cells/well) and incubated overnight. The next day, cells were washed and in each well media containing C-6-cHy-NPs was added. At different time intervals (0.5, 1, 2, 3, 4, and 5 h), the cells of a well were washed using PBS and analyzed under the fluorescence microscope using a 20× lens (Revolve, Discover Echo Inc., San Diego, CA, USA) at λex 460 nm. Counterstain Hoechst 33342 (1 μg/mL, 10 min incubation in the dark, λex 350 nm) was used.

### Cell Cytotoxicity Study

The HEI-OC1 cells were seeded in a 96-well plate and incubated overnight at 33° C. (5% CO₂). The treatment groups were kept as untreated control, CisPt only, CisPt+FL-cHy-NPs (20 μM equivalent to FL), CisPt+HK-NPs (20 μM equivalent to HK), CisPt+FL&HK-cHy-NPs (10 μM equivalent to FL&HK), and CisPt+STS (20 μM). First, 100 μL media containing 20 μM FL/HK/STS equivalent concentration of NPs or drugs were added to the respective wells. After 4 h of incubation, 100 μL media containing 50 μM CisPt was added. At the endpoint (48 h incubation), the cells were washed with fresh media two times and 100 μL media containing MTT reagent (0.5 mg/mL) were added to each well. After 4 h of incubation, the solubilization buffer (100 μL, DMSO:RIPA buffer; 50:50) was added to each well. After overnight incubation, the absorbance at 570 nm was recorded using UV-vis multi-well plate spectrophotometer (Synergy 2, BioTek, USA).

### Intracellular ROS Generation, Apoptosis, and Necrosis

The HEI-OC1 Cells (10000 cells/well in 96-well plates) were treated with various treatment groups as described above in the cell cytotoxicity study section. The endpoints for ROS generation, apoptosis, and necrosis studies were set up at 24, 36, and 48 h, respectively. For ROS generation study, the cells were washed two times with fresh media and then culture media containing DCFH₂DA reagent (10 μM) was added to each well. After 30 min incubation in dark at 33° C. (5% CO₂) the cells were washed with PBS three times. Fresh PBS (100 μL) was added and the fluorescence was recorded at λex/em 480/530 nm using a multi-well plate fluorescence spectrophotometer (Synergy 2, BioTek, USA). For apoptosis study, at the endpoint (24 h), the cells were washed three times and incubated with caspase 3/7 assay reagent according to the manufacturer's protocol (#APT423, CaspaTag™ In Situ Caspase-3/7 Detection Kit, Sigma-Aldrich, Sant Louis, MO, USA) for 30 min [37]. After washing, the fluorescence was recorded at λex/em 480/530 nm using a multi-well plate fluorescence spectrophotometer (Synergy 2, BioTek, USA). For analyzing the necrotic state of the cells, at the endpoint (48 h), the cells were washed using PBS and supplemented with fresh PBS containing PI (1 μg/mL). After 15 min incubation in dark, the fluorescence was recorded at λex/em 530/615 nm using a multi-well plate fluorescence spectrophotometer (Synergy 2, BioTek, USA). The fluorescence microscopy analysis of ROS generation, apoptosis, and necrosis experiments was performed. Briefly, nuclei of the cells from all experiments were counterstained using HOECHST-33342 (1 μg/mL) for 15 min in the dark. The cells were observed under the fluorescence microscope using a 20× lens at the respective dye fluorescence filter as described above. For HOECHST-33342 λex/em 350/460 nm fluorescence filter was used.

### Western Blot Analysis

After the treatment endpoint, in each well, cells were washed with PBS, and incubated with 100 μl mixture of 2× blue dye (90 μL, Recipe for 2× blue dye (10 mL): 0.5 M Tris HCl pH 6.8 (2.5 mL), 10% SDS (4 mL), Glycerol (2 mL), 0.1% Bromophenol Blue (0.3 mL), 2-mercaptoethanol (1 mL), DI water (0.2 mL).) and β-mercaptoethanol (10 μL) for 1 min at room temperature. The cells were collected using sterile cell scraper in microcentrifuge tube. The samples were centrifuged for 10 sec (1000 rpm) and heated for 5 min at 100° C. The samples were either loaded to the gel (SDS-PAGE) or stored at −20° C. for further use. After running the samples in precast SDS-PAGE gel (4-15% Mini-PROTEAN® TGX™ Precast Protein Gels, BIO-RAD) were transferred to the PVDF membrane using Trans-Blot® Turbo™ Transfer System (BIO-RAD). The membrane were blocked using nonfat dried milk (5%) in TBS-T containing Tween 20 (0.1%) at room temperature for 1 h. After washing, the membranes were incubated with respective assay primary antibodies; anti caspase 3 (#14220s, 1:1000) and anti cleaved caspase 3 (#9664s, 1:1000), (all from cell signaling technologies) diluted in nonfat dried milk (1.5%) in TBS-T, overnight at 4° C. The membranes were then washed using TBS-T (4 times every 5 min) and incubated further with goat anti-rabbit (#12004161, BIO-RAD, 1:2500) or goat anti-mouse (#92632210, Li-Cor, 1:2500) secondary antibodies for 1 h. The membranes were washed using TBS-T (4 times every 5 min). The bands corresponding to target expressions were imaged by Biorad's ChemiDoc Imaging System.

### Preparation of FL-cHy-NPs and HK-cHy-NPs Embedded Thermoresponsive Hydrogel Formulation

The thermoresponsive hydrogel, in non-limiting embodiments, comprises a mixture of a poloxamer and a carbomer. In one embodiment, the hydrogel may comprise a mixture of Poloxamer-407, Poloxamer-188, and Carbomer-940, which may be combined and mixed, fr example, at a concentration of 24, 15, and 0.1% w/v, respectively, in a cold (4° C.) colloidal solution of prepared NPs (FL-cHy-NPs and HK-cHy-NPs) and kept at 4° C. overnight. The thermoresponsive behavior of the prepared gels was characterized by keeping them at 25 and 37° C. The hydrogel formulations kept at 25 and 37° C. were instantly frozen using liquid nitrogen and then freeze-dried using a lyophilizer. The dried powder of the samples was spread on the carbon tape, fixed on the mounting stub, and sputtered with gold [39]. The processed samples were then analyzed using high-resolution FE-SEM; Neon 40EsB, Zeiss, Baden-Wurttemberg, Germany).

### In Vitro Drug Release Kinetics

Two 24 well plates were taken equipped with 6.5 mm inserts (Transwell®, Costar, Corning, NY, USA) having 8 m pores polycarbonate support membrane six in each plate. The developed hydrogel formulations containing FL-cHy-NPs and HK-cHy-NPs were filled in the inserts (n=3, in each plate and each formulation). One plate was placed at 37° C. while the other one was kept at 25° C. After 30 min, the receiver media (1 mL, PBS pH 7.4) was filled in each well-containing insert. Other wells were filled with deionized water to maintain humidity in the plate. The samples (100 μL) were drawn from the receiver well at different time intervals (0.5, 1, 3, 6, 10, 24, 48, 72, 144, and 312 h) and stored at 4° C. The drug content (FL and HK) was extracted using DCM; solvent-solvent extraction. After evaporating the DCM, the extracted FL and HK content was dissolved in methanol (100 μL, 100% LC-MS grade). The drug quantification of FL and HK in each sample was done using HPLC using the same procedure described above. The release kinetics of FL and HK from both hydrogels were calculated using the Korsmeyer-Peppas model (eq 7) [40].

$$\frac{{M}_{t}}{{M}_{∞}} = {k}_{1} {t}^{n} \qquad ( Eq . 6 )$$

where, Mt is the mass of the drug released at time t; M_{∞} is the mass of the total drug; k₁ is the structural and geometric characteristics of dosage form related constant; n is the release mechanism exponent.

### Otoprotection Studies on Zebrafish

Zebrafish were maintained at Creighton University Animal Resource Facilities by standard methods and per the Institutional Animal Care and Use Committee. Fish were maintained at 28.5° C. in E3 media with a 14-hour light/10-hour dark cycle. Otoprotection studies were performed in 5-6 dpf wild type (TuAB) fish as previously described [2]. Briefly, fish were pre-incubated for one hour in E3 media containing FL, HK or FL+HK and then co-incubated for 6 hours with the corresponding cytoprotective molecules and clinical cisplatin 400 mM. Concentrations were as follow: FL or HK=33 mM, 17 mM and 2 mM; FL+HK=17+17 mM, 8.5+8.5 mM and 3+3 mM. Fish were also incubated with empty NPs as control. After the treatment, fish were transferred to fresh E3 media for one hour to recover, followed by fixation (4% PFA) and processing for confocal imaging. For the detection of neuromast hair cells, fish were immunostained for the hair cell marker, otoferlin (HCS-1, DSHB, 1:200 dilution). Hair cells were manually counted using a Zeiss AxioSkop 2 fluorescence microscope. The neuromasts inspected were part of the cranial system and included the otic, middle, and opercular neuromasts. Ten to twelve fish were used per treatment. Confocal imaging was performed using a Zeiss LSM 700 confocal laser scanning image system. Images were captured at room temperature with automatically set sectioning and processed with ZEN black edition software. Z-stacks are presented as flat Z-projections. Final figures were assembled using Photoshop and Illustrator software (Adobe).

### Statistical Analysis

GraphPadPrizm 9 (San Diego, USA) was used for the statistical analysis of data through one-/two-way ANOVA. The tests were validated using Šidák's/Dunnet multiple comparison post-hoc test where p<0.05 was considered as significantly different. The graphs and figures were drawn using GraphPad Prizm 9, Origin Pro 9, Microsoft Office and Biorender.com.

### Results and Discussion

### Analytical Method Development for Analysis of Flunarizine (FL) and Honokiol (HK)

The HPLC-based analytical methods for qualitative and quantitative determination of FL and HK were successfully developed. The HPLC chromatogram showed retention times of 1.718 and 2.222 min for FL and HK, respectively with a total acquisition time of 4 min. The low LOD values were 0.059±0.013 and 0.038±0.004 μg/mL for FL and HK, respectively while the LOQ values were found to be as low as 0.179±0.040 μg/mL (for FL) and 0.116±0.012 μg/mL (for HK) (Tables 2 and 3). The standard equations for the quantitative determination of FL and HK in the samples were found to be ‘y=34.897x+1.553’ and ‘y=33.84x+1.5287’, respectively, where, y is the area of the curve and x is the concentration in g/mL units. The developed analytical method was found to be highly sensitive and robust for the qualitative and quantitative characterization of FL and HK in various samples during the study.

### DoE-CCD Based Formulation Parameters Optimization for the Synthesis of FL-cHy-NPs and HK-cHy-NPs

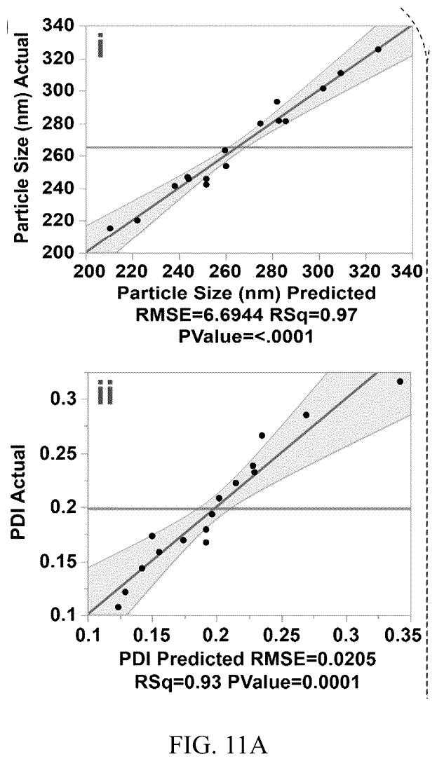

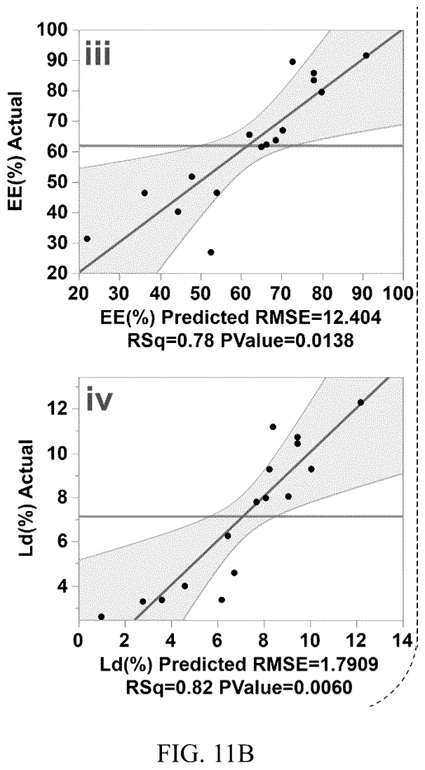

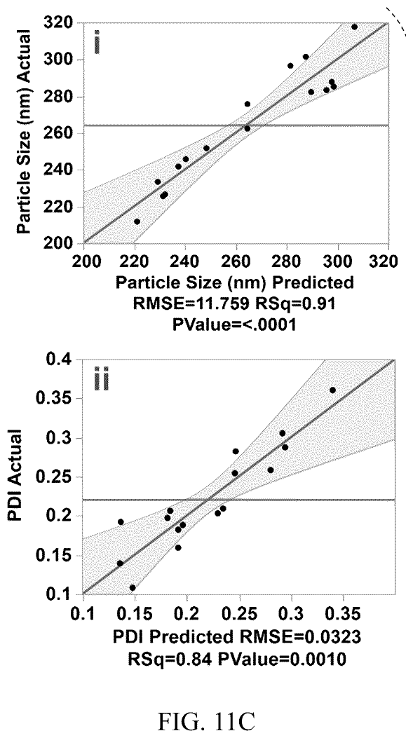

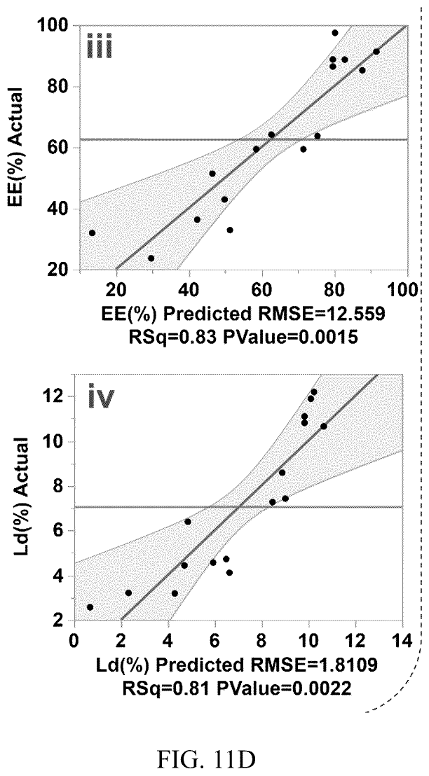

Prior to the final synthesis of the crosslinked NPs, the synthesis parameters were optimized using the DoE approach. A CCD was employed for the optimization of the three factors i.e. concentration of PPS-mPEG₂₀₀₀, PCDA, and drugs (FL or HK) with respect to four responses (size, PDI, EE(%), and dL(%)) for the synthesis of NPs. To optimize the synthesis of both FL-cHy-NPs and HK-cHy-NPs, the experiments (16 runs suggested by the software) with three different parameters namely PPS-mPEG concentration, PCDA concentration (% w/w of PPS-mPEG) and drug concentration (FL or HK; % w/w of PPS-mPEG and PCDA) were performed. The effect of various factors on the responses [particle size, PDI, encapsulation efficiency (EE(%)), and loading (Ld(%))] was screened by running the Standard Least Square model. The actual vs predicted value plots showed both null hypothesis (response independent of factors, horizontal line) and alternative hypothesis (response dependent on factors, slanted line) in the graphs (FIGS. 11A-11D).

The 95% confidence region was also present in each plot above the alternative hypothesis (slanted) line. Visually, in each response plot, the null hypothesis line (horizontal line) was not contained within the shaded region (95% significance) suggested the test model was significant. This was confirmed by the AVOVA analysis that suggest the F-test value for each plot was <0.05. Further, for a true fit, the lack of fit must be non-significant (F>0.05), visually, it was observed that the slanted line is going through the middle of the data points, and most of the response points present within the 95% confidence area near the slanted line. This observation suggested that the data was well fitting with the line and the lack of fit was not significant. This was confirmed by the Lack of Fit table of each response that showed F>0.05.

To further confirm the best fitting of the model, scatter index (SIn) values for each response were calculated using SEM and average values of each response. It was observed that the SIn values of each response were ≤25% which confirmed the CCD-based standard least square model could perform well for the factor optimization to get better responses in the NP development process (Table 6). Further, the analysis of residual vs. predicted plots showed the random distribution of the response values around the residual line (0-line) which suggests a good fit model for the respective response. Moreover, the studentized residual plot for each run (row) did not show significant outliers from the 95% simultaneous limits estimated by the Bonferroni test.

TABLE 6
The mean values of responses, RMSE, and SIn.

| | | Mean response values | | | | | |
|---|---|---|---|---|---|---|---|
| | | of all 16 runs | | RMSE | | SIn | |
| | | FL- | HK- | FL- | HK- | FL- | HK- |
| S. | | cHy- | cHy- | cHy- | cHy- | cHy- | cHy- |
| No. | Response | NPs | NPs | NPs | NPs | NPs | NPs |
| 1 | Particle | 265 | 264 | 6.69 | 11.76 | 2.5% | 4.4% |
| | Size (nm) | | | | | | |
| 2 | PDI | 0.199 | 0.221 | 0.02 | 0.032 | 10% | 15% |
| 3 | EE (%), | 61.9 | 62.68 | 12.4 | 12.56 | 20% | 20% |
| 4 | Ld (%) | 7.13 | 7.06 | 1.79 | 1.81 | 25% | 25% |

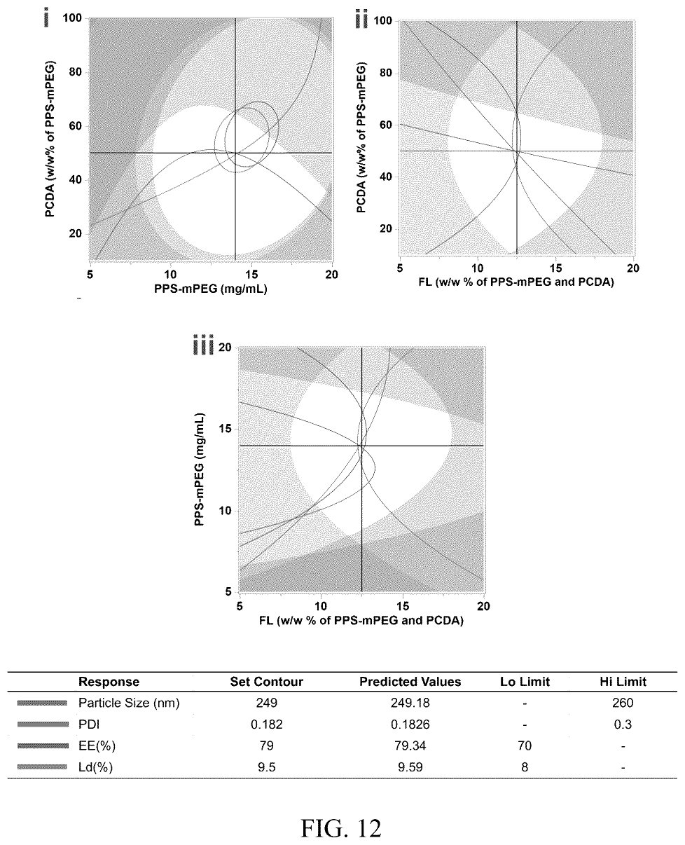

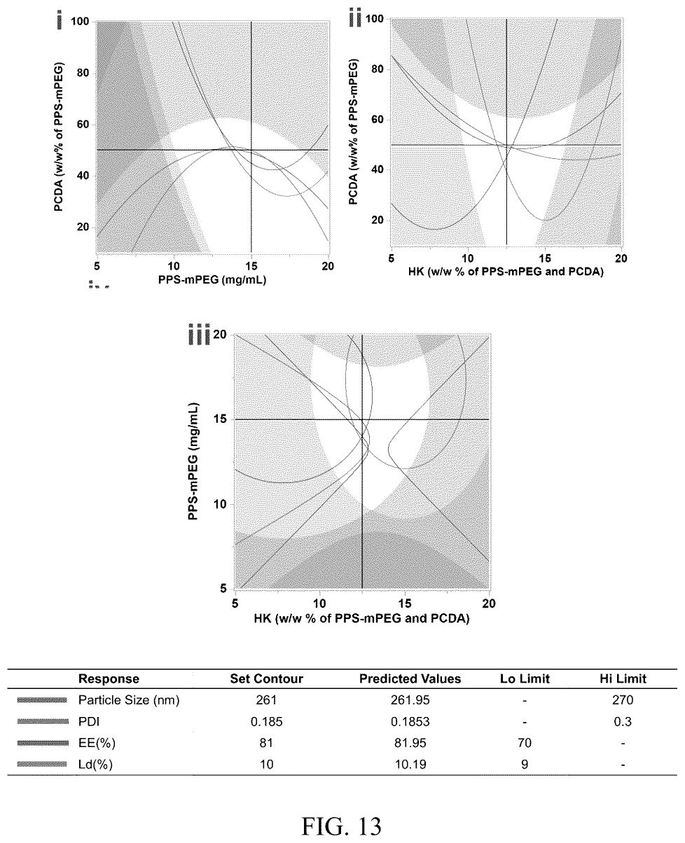

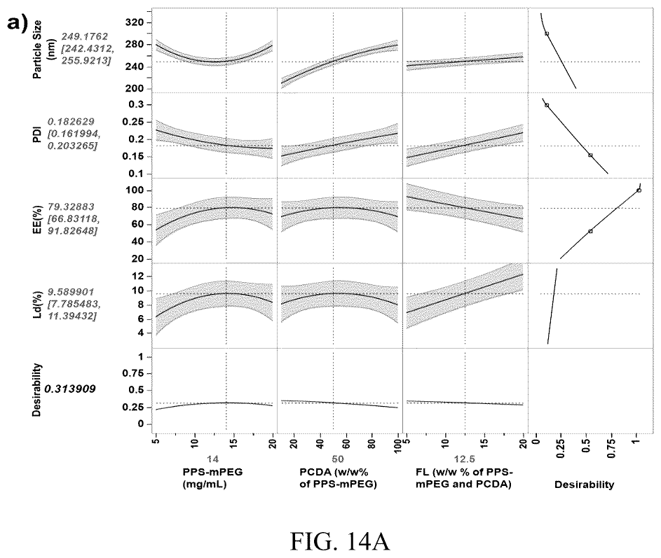

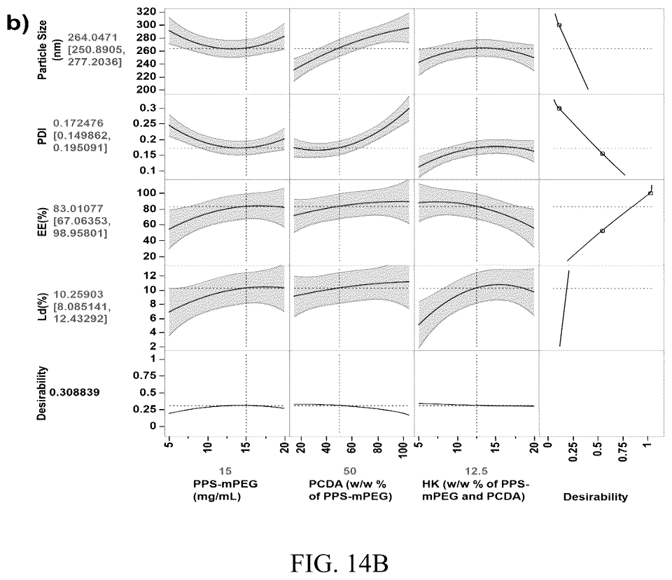

After ensuring the good fit model, the response contour graphs were plotted to determine the effect of various factors on the responses [particle size, PDI, encapsulation efficiency (EE(%)), and loading (Ld(%))]. By setting the contour near the predicted values for highest desirable response, contour graphs for mixture of all the responses were plotted. Using these plots, the highly favorable factors [(polymers; PCDA, PPS-mPEG, and drugs; FL and HK)] concentrations were obtained (FIGS. 12-13). Furthermore, the optimal factor conditions were confirmed using a prediction profiler graphical tool (FIGS. 14A-14B). The optimized concentrations of PPS-mPEG, PCDA, and FL were found to be 14 mg/mL, 50% (w/w of PPS-mPEG) and 12.5% (w/w of PPS-mPEG and PCDA), respectively, for the synthesis of FL-cHy-NPs with ˜250 nm size, 0.18 PDI, 79% EE and 9.6% Ld (FIG. 14A). Similarly, to synthesize HK-cHy-NPs with ˜260 nm size, 0.18 PDI, 82% EE and 10% Ld, the concentrations of PPs-mPEG, PCDA and HK, 15 mg/mL, 50% (w/w of PPS-mPEG) and 12.5% (w/w of PPS-mPEG and PCDA), respectively, were found to be optimal (FIG. 14B).

### Characterization of FL-cHy-NPs and HK-cHy-NPs

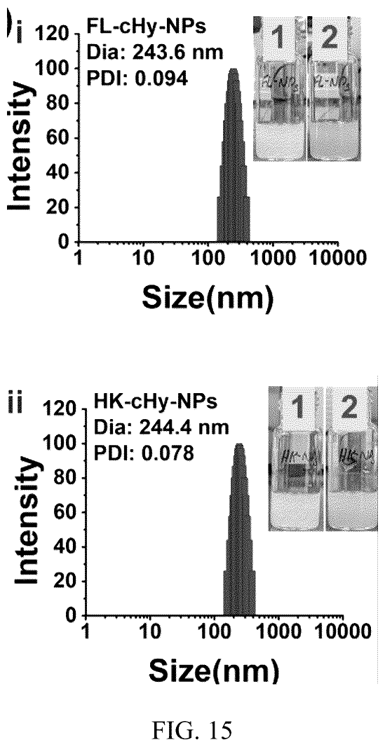

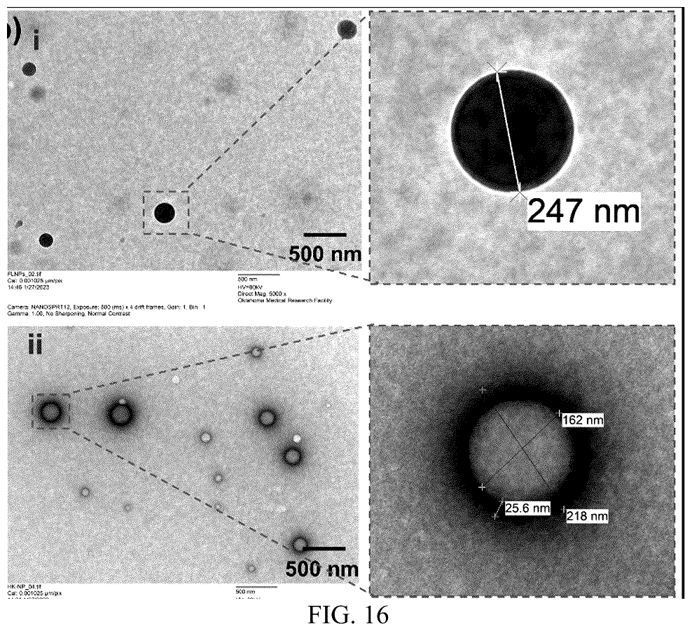

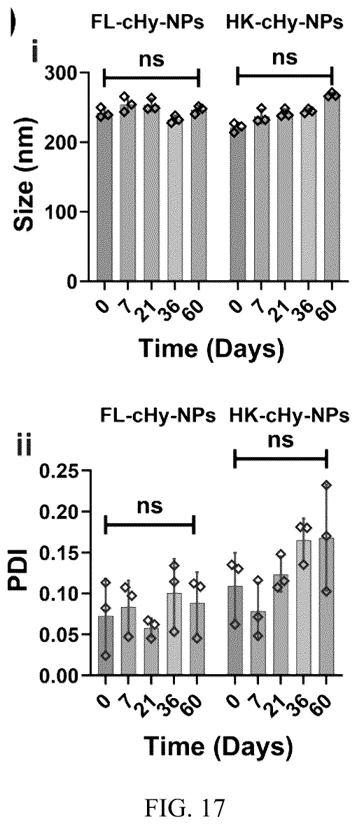

After preparation, the FL-cHy-NPs and HK-cHy-NPs were carefully characterized. The hydrodynamic diameter of the FL-cHy-NPs and HK-cHy-NPs determined using DLS was found to be 243.6±1.9 and 244.4±2.5 nm, respectively. The PDI values of the FL and HK-loaded cHy-PCDA-PPS-mPEG NPs were found to be 0.094±0.055 and 0.078±0.045, respectively (FIG. 15). These PDI values (≤0.3) suggest that the synthesized NPs were highly dispersed in the colloidal solution and were having uniform size distribution. The TEM analysis of both FL-cHy-NPs and HK-cHy-NPs confirmed the spherical morphology after the final preparation of the NPs (FIG. 16). The EE(%) of FL and HK in the respective NPs were found to be 78.3±8.1 and 80.52±8.4%, respectively. The Ld(%) of FL and HK in the respective NPs were found to be 9.47±1.21 and 9.73±1.41%, respectively. The size, PDI, encapsulation efficiency and loading values of the synthesized NPs were shown similar to the predicted values of prediction profiler. These results confirmed the validity of the obtained predicted factor values by CCD were feasible to synthesize FL-cHy-NPs and HK-cHy-NPs. Further, the stability of the NPs in the colloidal solution was found to be for more than 60 days at 4° C. (FIG. 17).

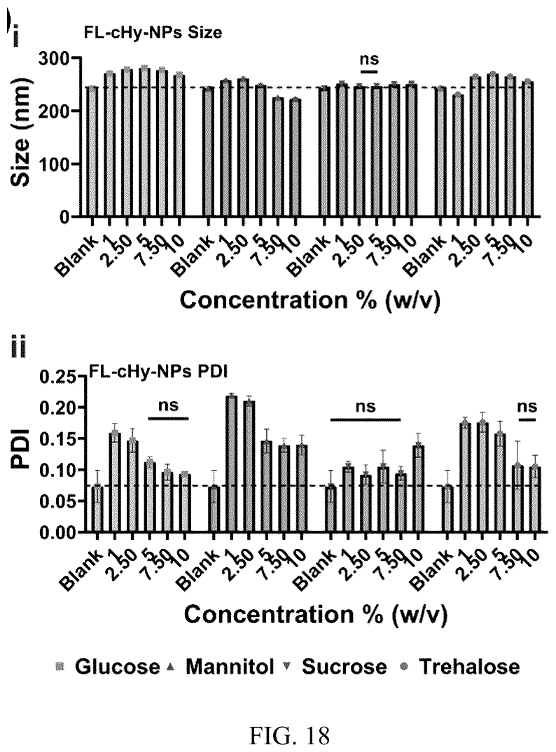

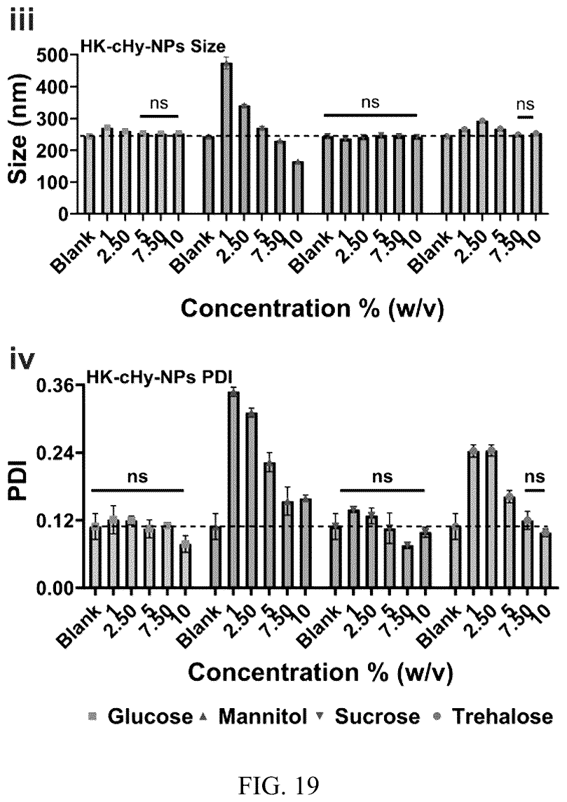

Although the prepared FL-cHy-NPs and HK-cHy-NPs were highly stable in the solution, their long-term storage is possible after lyophilization. Because, in the solution even the NPs were found to be highly stable morphologically, however, the drug release from the FL-cHy-NPs and HK-cHy-NPs can not be controlled. Therefore, the lyophilization study of the prepared FL-cHy-NPs and HK-cHy-NPs was done using different cryoprotectants (glucose, mannitol, sucrose, and trehalose) at their different concentrations (1, 2.5, 5, 7.5, and 10% w/v). After the reconstitution of NPs, the results suggested that sucrose and glucose showed relative stabilization potential as a cryoprotectant. Nevertheless, it did not show a significant protective effect at lower concentrations (<5% w/v). Other cryoprotectants (mannitol and trehalose) were not able to maintain appropriate size and PDI for the lyophilization of FL-cHy-NPs and HK-cHy-NPs. Therefore, sucrose possessed the cryoprotective effect at 2.5 and 5% w/v for the synthesized FL-cHy-NPs (FIG. 18) and HK-cHy-NPs (FIG. 19).

### Cellular Internalization Study

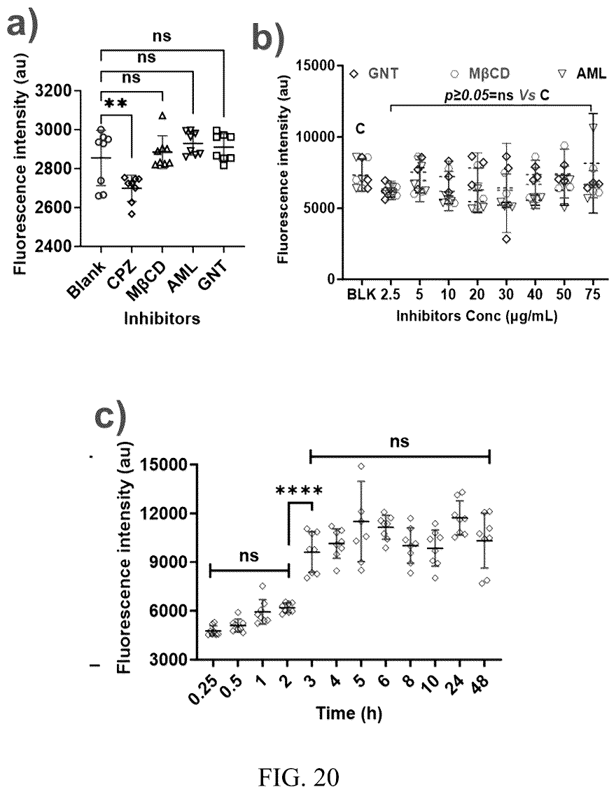

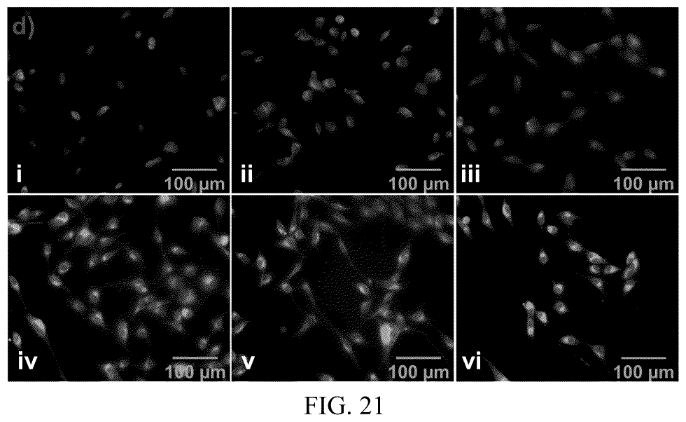

The cellular internalization of developed nanoparticles plays a crucial role in drug delivery in the cell cytoplasm and directly affects its pharmacological actions. The main pathways associated with the cellular internalization of various nanoparticles are Clathrin-mediated endocytosis, caveolin/cholesterol-dependent endocytosis, Macropinocytosis, and Caveolin mediated endocytosis, which can be inhibited by Chlorpromazine (CPZ), Methyl R Cyclodextrin (MβCD), Amiloride (AML), and Genistein (GNT), respectively. [41] To study the majorly associated pathway for cHy-NPs cell internalization, fluorescent-labeled (Coumarin-6; C6-loaded) cHy-NPs were used with or without pathway inhibitors. In the fluorescence spectroscopy analysis of the HEI-OC1 cells that were pretreated with MβCD, AML, and GNT, no significant change in the cellular internalization of C6-cHy-NPs was observed. However, in the CPZ-pretreated HEI-OC1 cells, significantly reduced cellular internalization of C6-cHy-NPs was observed (FIG. 20(a)). Therefore, it may be concluded that the pathway accompanying the cellular internalization of FL-cHy-NPs and HK-cHy-NPs was Clathrin-mediated endocytosis. These results corroborate the study of other PEGylated NP preparations those suggested that the PEGylate NP preparations mostly follow the Clathrin-mediated endocytosis as their main cellular internalization pathway [42]. To confirm the specific participation of the clathrin-mediated pathway and rule out the involvement of any other pathway, the study was repeated with various pathway inhibitors (MβCD, AML, and GNT) at different concentrations (2.5 to 75 μg/mL). There was no significant change in cellular internalization of C6-loaded NPs observed compared to inhibitor untreated cells even at higher inhibitor concentrations (FIG. 20(b)). Therefore, it was confirmed that Clathrin-mediated endocytosis was the main internalization pathway for cHy-NPs. Further, the time for the optimal cellular internalization of cHy-NPs was determined by recording the fluorescence emission at different time intervals. A notable increase in fluorescence intensity was observed up to 3 h of incubation after that there was no significant change noticed. Furthermore, the fluorescence intensity till 48 h with no critical changes confirmed the retention of NPs in the cells for a longer time (FIG. 20(c)). These results suggest that the efficient cellular internalization of cHy-NPs in the HEI-OC1 cells can be achieved within 3 h of incubation. These findings were further confirmed using fluorescence microscopy analysis of the HEI-OC1 cells. Similar to fluorescence spectroscopy, a significant increase in fluorescence was observed until 3 h of incubation and after that, there was no significant change in the fluorescence intensity in the cells was observed in microscopy images (FIG. 21). Moreover, the results of fluorescence microscopy and spectroscopy suggest that the NPs did not expel out from the cells over time as there was no significant change in the fluorescence was observed until 5 h of incubation. Overall, these results demonstrate that the developed cHy-NPs could efficiently deliver the loaded drugs (FL and HK) in the inner ear hair cells within 3-6 h of administration.

### Cisplatin-Induced Cytotoxicity Protection

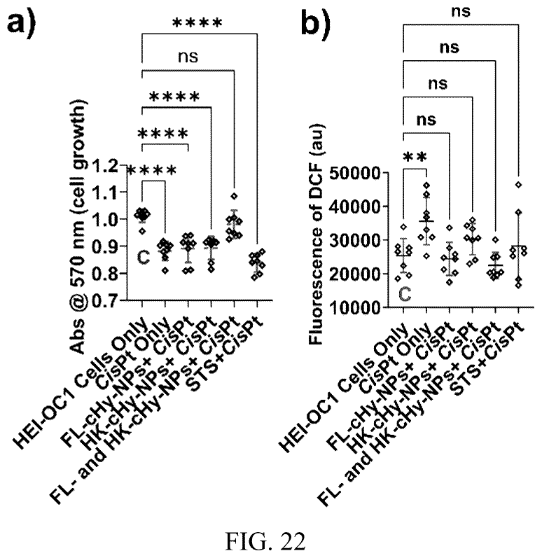

Cisplatin (CisPt) induces cell apoptosis and necrosis in the inner ear hair cells leading to permanent hearing loss [43,44]. We investigated the cytoprotective effect of FL-cHy-NPs and HK-cHy-NPs against CisPt-induced cytotoxicity on HEI-OC1 cells. The results suggested that the combination of FL-cHy-NPs and HK-cHy-NPs showed a significant cytoprotective effect compared to FL-cHy-NPs and HK-cHy-NPs alone. It was also observed that the cytoprotective effect of FL-cHy-NPs and HK-cHy-NPs against CisPt-induced cytotoxicity was even higher than the recently approved treatment regimen sodium thiosulfate (STS) [45,46] (FIG. 22(a)). The considerable cytoprotective effect might be achieved due to the ROS detoxification effect related to the cellular deacetylase (sirtuin 3; Sirt-3) activation in the presence of HK [47]. Moreover, a combined effect associated with the effect of FL was expectedly played a substantial role in cytoprotecting via activation of heme oxygenase-1 (HO-1) through Nrf2 mediated transcription [48]. Overall, enhanced cytoprotecting action against CisPt-induced cytotoxicity in HEI-OC1 (inner ear hair cells) can be achieved by co-delivering FL-cHy-NPs and HK-cHy-NPs.

### Inhibitory Effects of Intracellular ROS Generation, Apoptosis and Necrosis

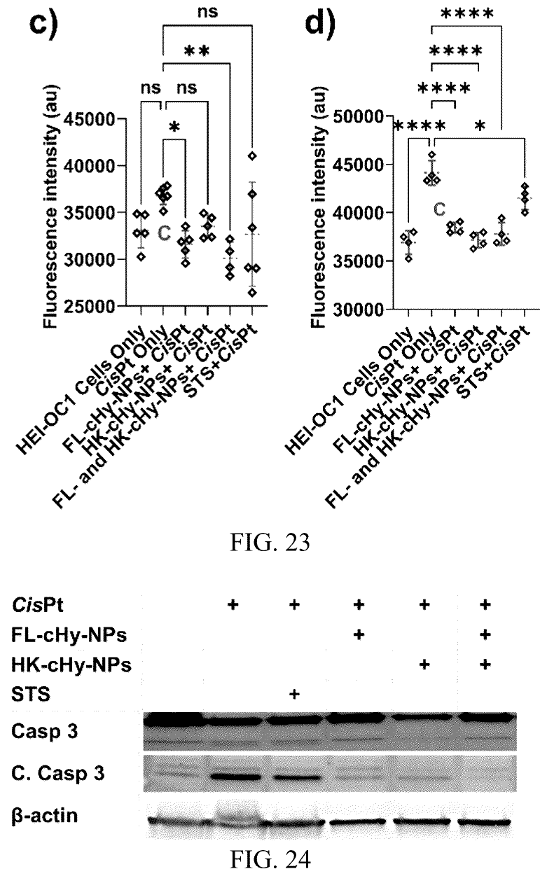

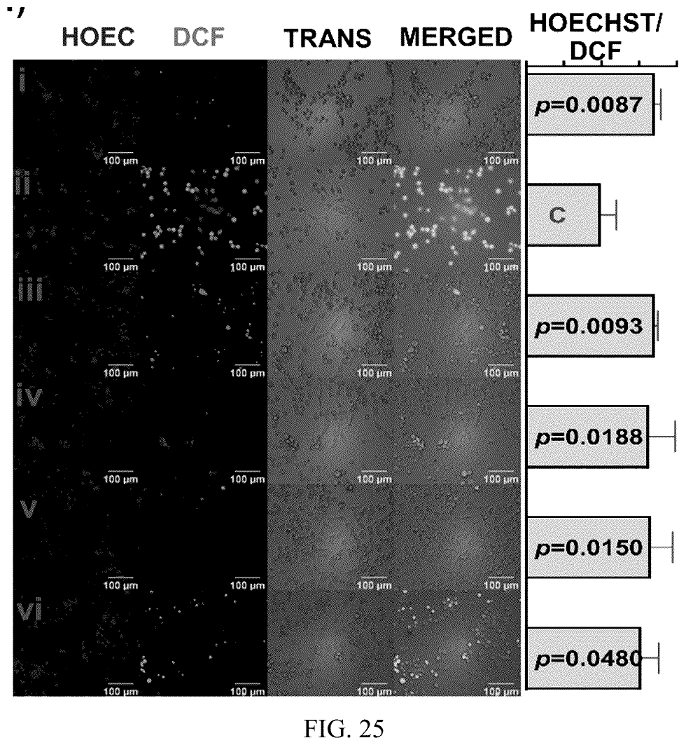

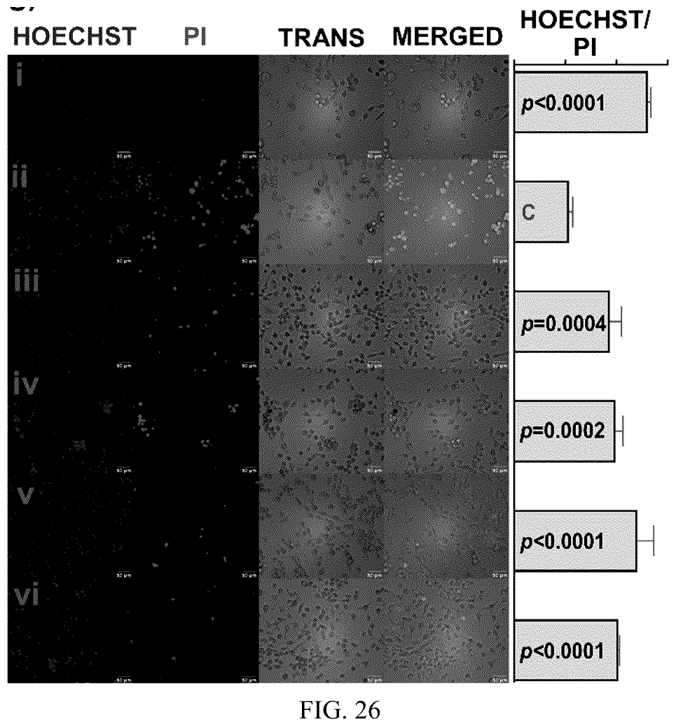

The high concentration of ROS in the cells may lead to creating oxidative stress in the cells. Therefore, we investigated the generation of intracellular ROS in different treatment groups using the DCFH₂-DA assay method. After 24 h of incubation (post CisPt addition), the untreated HEI-OC1 cells showed significantly lowered ROS generation compared to CisPt only-treated cells. The treatment groups with FL-cHy-NPs and HK-cHy-NPs (alone or in combination) and STS did not show a significant ROS generation compared to CisPt-only treated cells ((FIG. 22(a), FIG. 25). Furthermore, the MitoSOX assay was employed to confirm the quenching effect of ROS generation. The result of the study suggested that the cells those were treated with FL-cHy-NPs, HK-cHy-NPs and STS showed significantly reduced mitochondrial superoxide compared to CisPt-only treated cells ((FIG. 23(c)). After determining the effect of FL-cHy-NPs, HK-cHy-NPs and STS on CisPt-induced ROS generation in the cells, their significance on cell cycle and survival was further investigated. The cellular apoptosis study was done using a caspase 3/7 assay. The activation of caspase 3/7 was considered the confirmatory marker of the apoptotic stage of the cells (the higher the activation the higher the apoptosis). The results of the caspase-3/7 assay suggested that the cells that were treated with the FL-cHy-NPs and HK-cHy-NPs (alone or in combination) and STS showed significantly reduced signals of caspase 3/7 activation compared to the CisPt only treated cells ((FIG. 23(d)). Moreover, there was significant reduction in cleaved caspase 3 proteins were observed in the FL-cHy-NPs and HK-cHy-NPs treatment groups, alone or in combination. Interestingly, a significant reduction of cleaved caspase-3 was observed in the combination treatment as compared to the groups that were treated alone. These results, in corroboration with cell cytotoxicity and apoptosis study data, confirm that the combination of FL-cHy-NPs and HK-cHy-NPs provides better protection against CisPt induced toxicity ((FIG. 24). Therefore, it may be concluded that the developed FL-cHy-NPs and HK-cHy-NPs were efficiently able to protect the HEI-OC1 cells from CisPt-induced cytotoxicity by quenching ROS generation and the subsequent reduction of oxidative stress which were responsible for the activation of apoptotic pathways. Finally, the cytoprotective effect of the developed FL-cHy-NPs and HK-cHy-NPs was investigated by determining cell necrosis. The PI staining assay was done to confirm the cytoprotective effect of developed FL-cHy-NPs, HK-cHy-NPs after the treatment with FL-cHy-NPs, HK-cHy-NPs, STS and CisPt. The cells were stained with HOECHST-33342 and PI stains after 48 h of treatment. Compared to the untreated cells, a significant necrotic cell population was observed in the group treated with the CisPt only. However, cell groups that were treated with FL-cHy-NPs and HK-cHy-NPs (alone or in combination) and STS ((FIG. 26) prior to the CisPt exposure, did not show significant necrotic cell population. Therefore, our investigation confirmed that the cells treated with FL-cHy-NPs and HK-cHy-NPs (alone or in combination) showed a significant cytoprotective effect against CisPt-induced cytotoxicity that was comparable with the approved STS treatment.

### FL-NPs and HK-NPs Embedded Thermoresponsive Hydrogel Formulation

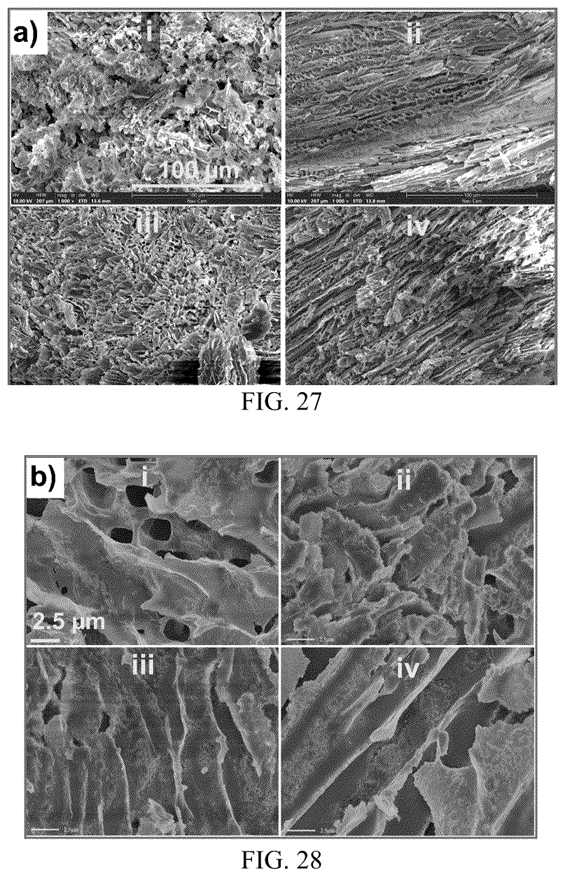

In order to achieve sustained delivery of FL and HK-NPs at the desired site of action, the thermoresponsive hydrogel formulation was prepared using our previous NanoSensoGel technology [49]. The developed thermoresponsive hydrogel was present in sol state at room temperature (25° C.), however, at body temperature (37° C.) it converted in gel state. This desired feature was expected to achieve retention of the formulation at the round window membrane and for sustained release of the nanoparticles. Characteristically, the micellar rearrangement of polymeric units of poloxamer 407 and 188 in the gel had to be responsible for the formation of gel state at higher temperature [50,51]. The SEM analysis of hydrogels kept at 37° C. before freeze drying showed more arranged honeycomb patterns compared to the hydrogel that was kept at 25° C. ((FIG. 27). These results confirmed the structural changes in the hydrogel morphology related to temperature associated conformational changes in polymeric backbones. The entrapment of NPs in the developed hydrogel formulation was further evaluated ((FIG. 28) utilizing the high resolution focused-emission scanning electron microscopy images for a quantitative and qualitative comparison by artificial intelligence tool. The FL-cHy-NPs and HK-cHy-NPs in the thermoresponsive hydrogels were segmented via a deep learning model created in the DigiM I2S software platform, then particle sizing statistics were computed. Quantification of particle size from 2D images presents challenge as things such as perspective artifacts, objects occluding others, and low contrast all contribute to reducing the accuracy of a segmentation. Size characterization in a 2D image is fundamentally limited by the lack of the 3rd dimension, because of this size distributions are only an estimation. To fully characterize these particles 3D analysis would be needed, using techniques such as Focused Ion Beam Scanning Electron Microscopy (FIB-SEM). The size distribution reported in Table 2 at D10, D50, and D90 were in relative agreement of the TEM image analysis.

TABLE 7
Size distribution analysis at D10, D 50, and D 90

| Size Distribution (μm) | D10 | D50 | D90 |
|---|---|---|---|
| HK-cHy-NPs 37° C. | 0.26 | 0.47 | 0.81 |
| FL-cHy-NPs 37° C. | 0.26 | 0.48 | 0.75 |
| HK-cHy-NPs 25° C. | 0.29 | 0.51 | 0.86 |
| FL-cHy-NPs 25° C. | 0.10 | 0.22 | 0.36 |

Release of FL and HK from the Hydrogel Formulation

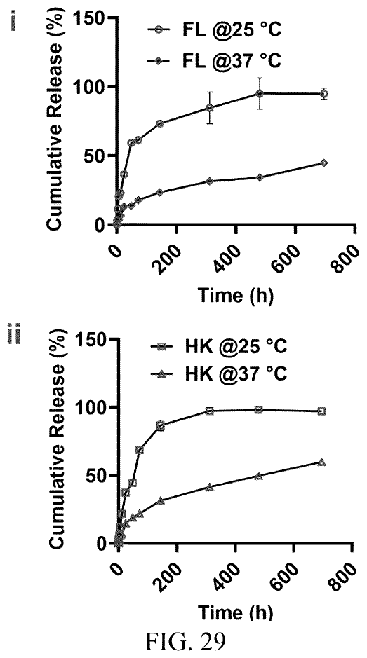

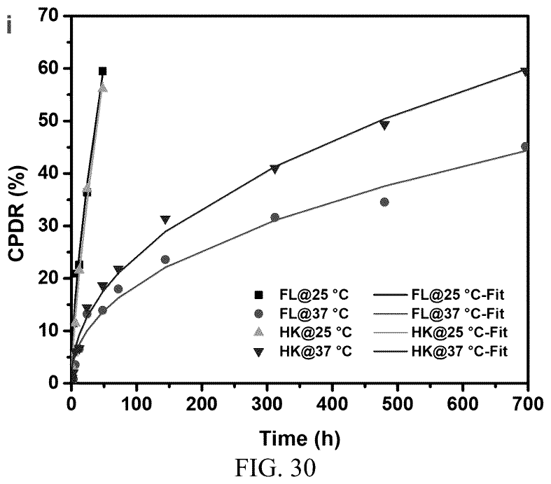

The desirable amount of the drugs should be released from the formulation to achieve anticipated pharmacodynamic effect at the targeted site. Therefore, the release study of FL and HK from the respective hydrogel formulation was done at 25 and 37° C., using PBS (pH 7.4) as the receiving media. At 25° C., the release of FL and HK was found to be higher as compared to 37° C. The maximum release of FL and HK from the hydrogel was found to be ˜50% in one month at 37° C., however, ˜60% release of FL and HK was observed in 48 h at 25° C. ((FIG. 29 (upper and lower panels)). The release kinetics of the formulation was determined by Korsmeyer-Peppas model (KP-model, (FIG. 30). It was observed that the cumulative percent drug release (CPDR) values fits well with the KP-model. The release mechanism values (n) of 25° C. data of FL and HK were found to be between 0.5 to 1 which suggested non-Fickian diffusion mechanism (0.5<n<1) [52]. However, the n values of FL and HK 37° C. release study were found to be <0.5 which suggested the Fickian release mechanism (Table 8). Since the FL and HK were loaded in the NPs, the mechanism of the release is mainly depending on the release of the FL/HK-cHy-NPs from the hydrogel formulations. At 27° C., the release kinetics follow non-Fickian release because the NPs diffuse from the gels because of the combination of polymer swelling as well as normal diffusion [53]. However, at 37° C., the hydrogel formulation present in gel form. In this case the swelling effect could be nullified and the NPs release mechanism follow the Fickian diffusion kinetics which provides sustained release of the drugs.

TABLE 8
The Korsmeyer-Peppas model fit values of the FL
and HK release from the developed formulations

| | |
|---|---|
| FL-cHy-NPs | HK-cHy-NPs |

| Model parameters | 25° C. | 37° C. | 25° C. | 37° C. |
|---|---|---|---|---|
| K₁ | 5.14 | 2.46 | 3.37 | 2.92 |
| n | 0.63 | 0.44 | 0.73 | 0.46 |
| R² | 0.9921 | 0.9916 | 0.9976 | 0.9968 |

### Screening of FL-cHy-NPs and HK-cHy-NPs

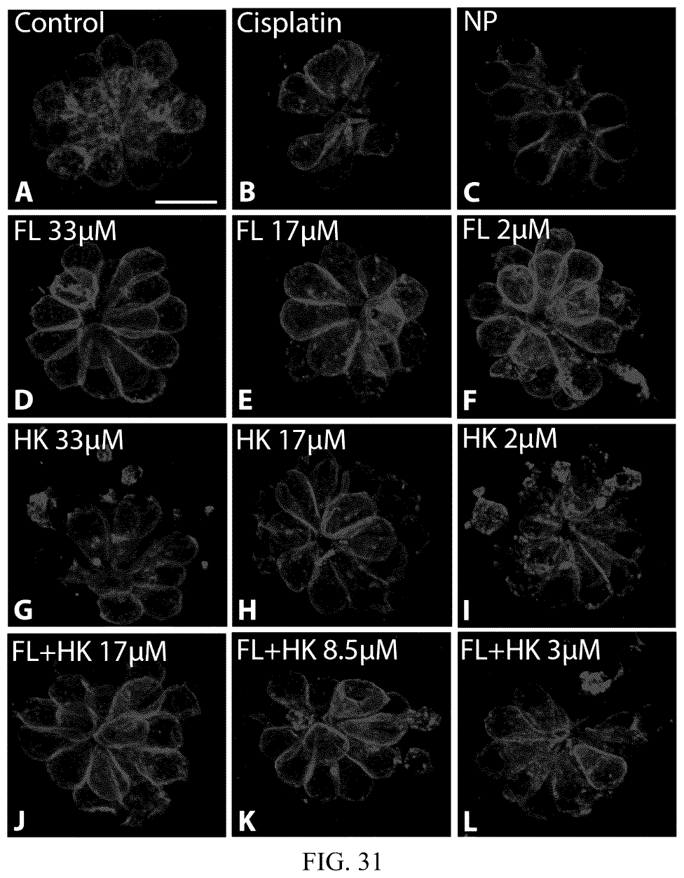

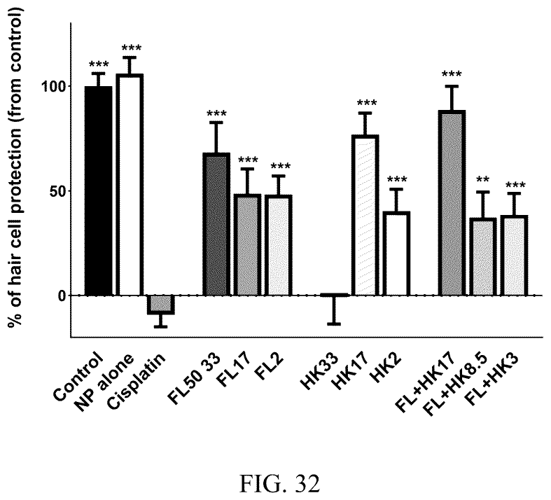

We used zebrafish as an in vivo model to test the therapeutic potential of FL and HK. FIG. 31 shows that when fish was incubated with the individual compounds or in combination, neuromast hair cells were protected against cisplatin-induced ototoxicity. We observed a significant decrease in their numbers in the cisplatin-only group compared to control (b) vs. (a). Conversely, treatment with FL, HK or FL+HK, prevented hair cell loss (d-1). Additionally, the incubation with empty nanoparticles (NPs), (c) did not have any effect. FIG. 32 shows quantification of the number of hair cells per neuromast in the results of FIG. 31. Results are expressed as percentage of protection, with 100% representing control animals and 0% cisplatin-treated fish. Overall, these results demonstrate that FL and HK can prevent or reduce cisplatin-induced hearing loss.

### Discussion

In the present work, the inner-ear targeted cross-linked hybrid nanoparticles embedded in thermoresponsive hydrogel are intended to provide a sustained therapeutic drug release in a drug-induced cytotoxic environment. In non-limiting examples, the flunarizine (FL)- and honokiol (HK)-loaded cHy-PCDA-PPS-mPEG-nanoparticles (cHy-NPs) were successfully optimized using design of experiment-central composite design and synthesized using ultrasonication assisted single emulsion nanoprecipitation solvent evaporation technique. In other embodiments, the compounds Amifostine, Sodium Thiosulfate (STS), STS-IV, Bucilamine, Diltiazem, Dexamethasone, N-acetylcysteine, Ebselen, Agmatine, and Allicin may be used herein as the therapeutic drug component in the present compositions. The therapeutic drug used in the compositions herein may be a T-type calcium channel blocker.

In at least certain embodiments, the amount of PCDA in relation to PPS-mPEG in the drug-cHy-NP formulation is about 50% to 60%. In certain embodiments, the PCDA:PPS-mPEG ratio may be in a range from about 1:2 w/w to about 3:5 w/w. Where the endpoint of the range is about 1:2 w/w, the endpoint of the range may be 1(±1%):2 w/w; or 1(±2%):2 w/w; or 1(±3%):2 w/w; or 1(±4%):2 w/w; or 1(±5%):2 w/w; or 1(±6%):2 w/w; or 1(±7%):2 w/w; or 1(±8%):2 w/w; or 1(±9%):2 w/w; or 1(±10%):2 w/w; or 1(±11%):2 w/w; or 1(±12%):2 w/w. Where the endpoint of the range is about 3:5 w/w, the endpoint of the range may be 3(±1%):5 w/w; or 3(±2%):5 w/w; or 3(±3%):5 w/w; or 3(±4%):5 w/w; or 3(±5%):5 w/w; or 3(±6%):5 w/w; or 3(±7%):5 w/w; or 3(±8%):5 w/w; or 3(±9%):5 w/w; or 3(±10%):5 w/w; or 3(±11%):5 w/w; or 3(±12%):5 w/w.

The standard least square model was found to be good fit for determining the factor affecting the synthesis of FL-cHy-NPs and HK-cHy-NPs. The good fit of the model for each response was confirmed by ANOVA (P<0.05), Lack of Fit (P>0.05), Actual Vs. Predicted plots, scatter index (SIn≤25%), Residual Vs. Actual response plots and Studentized Fit plots. The optimal factors to obtain desired response were determined using contour and prediction profiler graphs. For the synthesis of FL-cHy-NPs, the optimal concentrations of mPEG₂₀₀₀, PCDA and FL were found to be 14 mg/mL, 50% w/w of mPEG₂₀₀₀, and 12.5% w/w of ‘mPEG₂₀₀₀ & PCDA’, respectively, and to synthesize HK-cHy-NPs, the optimized concentrations of mPEG₂₀₀₀, PCDA and HK were found to be 15 mg/mL, 50% w/w of mPEG₂₀₀₀, and 12.5% w/w of ‘mPEG₂₀₀₀ & PCDA’, respectively. The optimized ‘size and PDI’ values of synthesized FL-cHy-NPs and HK-cHy-NPs were found to be ‘243.6±1.9 nm and 0.094±0.055’, and ‘244.4±2.5 nm and 0.078±0.045’, respectively. The ‘encapsulation and loading’ efficiencies of FL and HK in the respective NPs were found to be ‘78.3±8.1 and 9.47±1.21%’ and ‘80.52±8.4 and 9.73±1.41%’, respectively. The synthesized NPs were found to be stable in solution for at least two months at 4° C. The best cryoprotectant was sucrose (2.5-5%) for lyophilization. The C-6 NPs were able to internalized by the cells via clathrin mediated endocytosis process. The FL-cHy-NPs and HK-cHy-NPs in combination was able to protect cytotoxic effect of cisplatin in HEI-OC1 cells. It was also found that the FL-cHy-NPs and HK-cHy-NPs alone or in combination have ROS quenching and significantly low apoptosis/necrosis effect in CisPt treated HEI-OC1 cells. The thermoresponsive hydrogel was synthesized by Poloxamer 407/Poloxamer 188/Carbomer 940 based hydrogel. The FL-cHy-NPs and HK-cHy-NPs were embedded in the hydrogel formulation showed sustained release of the FL and HK for 30 days at 37° C. Hence, the cross-linked hybrid nanoformulation demonstrates an effective approach to protect the inner ear hair cells from the cytotoxic environment of the ototoxic drugs.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus, the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

### REFERENCES

[1] P. S. Steyger, Mechanisms of Ototoxicity and Otoprotection, Otolaryngol. Clin. North Am. 54 (2021) 1101-1115. https://doi.org/10.1016/j.otc.2021.08.007.
[2] J. Kim, S. Hemachandran, A. G. Cheng, A. J. Ricci, Identifying targets to prevent aminoglycoside ototoxicity, Mol. Cell. Neurosci. 120 (2022) 103722. https://doi.org/10.1016/j.mcn.2022.103722.
[3] L. P. Rybak, V. Ramkumar, D. Mukherjea, Ototoxicity of Non-aminoglycoside Antibiotics, Front. Neurol. 12 (2021) 652674. https://doi.org/10.3389/FNEUR.2021.652674/BIBTEX.
[4] C. Lanvers-Kaminsky, A. am Zehnhoff-Dinnesen, R. Parfitt, G. Ciarimboli, Drug-induced ototoxicity: Mechanisms, Pharmacogenetics, and protective strategies, Clin. Pharmacol. Ther. 101 (2017) 491-500. https://doi.org/10.1002/cpt.603.
[5] A. Chattaraj, M. P. Syed, C. A. Low, T. K. Owonikoko, Cisplatin-Induced Ototoxicity: A Concise Review of the Burden, Prevention, and Interception Strategies, JCO Oncol. Pract. 19 (2023) 278-283. https://doi.org/10.1200/op.22.00710.
[6] A. Callejo, L. Sedó-Cabezón, I. D. Juan, J. Llorens, Cisplatin-induced ototoxicity: Effects, mechanisms and protection strategies, Toxics. 3 (2015) 268-293. https://doi.org/10.3390/toxics3030268.
[7] L. P. Rybak, D. Mukherjea, V. Ramkumar, Mechanisms of Cisplatin-Induced Ototoxicity and Prevention, Semin. Hear. 40 (2019) 197-204. https://doi.org/10.1055/s-0039-1684048.
[8] J. G. Gurney, J. K. Bass, A. Onar-Thomas, J. Huang, M. Chintagumpala, E. Bouffet, T. Hassall, S. Gururangan, J. A. Heath, S. Kellie, R. Cohn, M. J. Fisher, A. P. Panandiker, T. E. Merchant, A. Srinivasan, C. Wetmore, I. Qaddoumi, C. F. Stewart, G. T. Armstrong, A. Broniscer, A. Gajjar, Evaluation of amifostine for protection against cisplatin-induced serious hearing loss in children treated for average-risk or high-risk medulloblastoma, Neuro. Oncol. 16 (2014) 848-855. https://doi.org/10.1093/neuonc/not241.
[9] D. R. Freyer, L. Chen, M. D. Krailo, K. Knight, D. Villaluna, B. Bliss, B. H. Pollock, J. Ramdas, B. Lange, D. Van Hoff, M. L. VanSoelen, J. Wiernikowski, E. A. Neuwelt, L. Sung, Effects of sodium thiosulfate versus observation on development of cisplatin-induced hearing loss in children with cancer (ACCL0431): a multicentre, randomised, controlled, open-label, phase 3 trial, Lancet Oncol. 18 (2017) 63-74. https://doi.org/10.1016/S1470-2045(16)30625-8.
[10] P. R. Brock, R. Maibach, M. Childs, K. Rajput, D. Roebuck, M. J. Sullivan, V. Laithier, M. Ronghe, P. Dall'Igna, E. Hiyama, B. Brichard, J. Skeen, M. E. Mateos, M. Capra, A. A. Rangaswami, M. Ansari, C. Rechnitzer, G. J. Veal, A. Covezzoli, L. Brugieres, G. Perilongo, P. Czauderna, B. Morland, E. A. Neuwelt, Sodium Thiosulfate for Protection from Cisplatin-Induced Hearing Loss, N. Engl. J. Med. 378 (2018) 2376-2385. https://doi.org/10.1056/nejmoa1801109.
[11] Z. Sarafraz, A. Ahmadi, A. Daneshi, Transtympanic injections of N-acetylcysteine and dexamethasone for prevention of cisplatin-induced ototoxicity: Double blind randomized clinical trial, Int. Tinnitus J. 22 (2018) 40-45. https://doi.org/10.5935/0946-5448.20180007.
[12] G. Simsek, B. M. Taş, N. B. Muluk, M. Azman, R. Kiliç, Comparison of the protective efficacy between intratympanic dexamethasone and resveratrol treatments against cisplatin-induced ototoxicity: an experimental study, Eur. Arch. Oto-Rhino-Laryngology. 276 (2019) 3287-3293. https://doi.org/10.1007/s00405-019-05635-x.
[13] S. Mohan, B. J. Smyth, A. Namin, G. Phillips, M. A. Gratton, Targeted amelioration of cisplatin-induced ototoxicity in guinea pigs, Otolaryngol.—Head Neck Surg. (United States). 151 (2014) 836-839. https://doi.org/10.1177/0194599814544877.
[14] M. Yildirim, H. M. Inançli, B. Samanci, M. F. Oktay, M. Enöz, I. Topçu, Preventing cisplatin induced ototoxicity by N-acetylcysteine and salicylate., Kulak Burun Bogaz Ihtis. Derg. 20 (2010) 173-183. https://pubmed.ncbi.nlm.nih.gov/20626325/(accessed Sep. 22, 2023).
[15] M. G. Riga, L. Chelis, S. Kakolyris, S. Papadopoulos, S. Stathakidou, E. Chamalidou, N. Xenidis, K. Amarantidis, P. Dimopoulos, V. Danielides, Transtympanic injections of N-acetylcysteine for the prevention of cisplatin-induced ototoxicity: A feasible method with promising efficacy, Am. J. Clin. Oncol. Cancer Clin. Trials. 36 (2013) 1-6. https://doi.org/10.1097/COC.0b013e31822e006d.
[16] S. J. Kim, C. Park, A. L. Han, M. J. Youn, J. H. Lee, Y. Kim, E. S. Kim, H. J. Kim, J. K. Kim, H. K. Lee, S. Y. Chung, H. So, R. Park, Ebselen attenuates cisplatin-induced ROS generation through Nrf2 activation in auditory cells, Hear. Res. 251 (2009) 70-82. https://doi.org/10.1016/J.HEARES.2009.03.003.
[17] H. S. So, H. J. Kim, J. H. Lee, J. H. Lee, S. Y. Park, C. Park, Y. H. Kim, J. K. Kim, K. M. Lee, K. S. Kim, S. Y. Chung, W. C. Jang, S. K. Moon, H. T. Chung, R. K. Park, Flunarizine induces Nrf2-mediated transcriptional activation of heme oxygenase-1 in protection of auditory cells from cisplatin, Cell Death Differ. 13 (2006) 1763-1775. https://doi.org/10.1038/sj.cdd.4401863.
[18] Y. Zhang, Z. Lv, Q. He, Agmatine Alleviates Cisplatin-Induced Ototoxicity by Activating PI3K/AKT Signaling Pathway, ENeuro. 9 (2022). https://doi.org/10.1523/ENEURO.0434-21.2022.
[19] X. Tan, Y. Zhou, A. Agarwal, M. Lim, Y. Xu, Y. Zhu, J. O'Brien, E. Tran, J. Zheng, D. Gius, C.-P. Richter, Systemic application of honokiol prevents cisplatin ototoxicity without compromising its antitumor effect, Am. J. Cancer Res. 10 (2020) 4416-4434. http://www.ncbi.nlm.nih.gov/pubmed/33415008%0Ahttp://www.pubmedcentral.nih.gov/articlere nder.fcgi?artid=PMC7783741 (accessed Sep. 22, 2023).
[20] J. Cai, X. Wu, X. Li, C. Ma, L. Xu, X. Guo, J. Li, H. Wang, Y. Han, Allicin Protects against Cisplatin-Induced Stria Vascularis Damage: Possible Relation to Inhibition of Caspase-3 and PARP-1-AIF-Mediated Apoptotic Pathways, Orl. 81 (2019) 202-214. https://doi.org/10.1159/000500557.
[21] R. A. Hazlitt, J. Min, J. Zuo, Progress in the Development of Preventative Drugs for Cisplatin-Induced Hearing Loss, J. Med. Chem. 61 (2018) 5512-5524. https://doi.org/10.1021/acs.jmedchem.7b01653.
[22] Q. Tang, X. Wang, H. Jin, Y. Mi, L. Liu, M. Dong, Y. Chen, Z. Zou, Cisplatin-induced ototoxicity: Updates on molecular mechanisms and otoprotective strategies, Eur. J. Pharm. Biopharm. 163 (2021) 60-71. https://doi.org/10.1016/j.ejpb.2021.03.008.
[23] FDA approves sodium thiosulfate to reduce the risk of ototoxicity associated with cisplatin in pediatric patients with localized, non-metastatic solid tumors|FDA, (n.d.). https://www.fda.gov/drugs/resources-information-approved-drugs/fda-approves-sodium-thiosulfate-reduce-risk-ototoxicity-associated-cisplatin-pediatric-patients (accessed Nov. 23, 2022).
[24] H. S. So, C. Park, H. J. Kim, J. H. Lee, S. Y. Park, J. H. Lee, Z. W. Lee, H. M. Kim, F. Kalinec, D. J. Lim, R. Park, Protective effect of T-type calcium channel blocker flunarizine on cisplatin-induced death of auditory cells, Hear. Res. 204 (2005) 127-139. https://doi.org/10.1016/j.heares.2005.01.011.
[25] H. S. So, H. J. Kim, Y. Kim, E. Kim, H. O. Pae, H. T. Chung, H. J. Kim, K. B. Kwon, K. M. Lee, H. Y. Lee, S. K. Moon, R. Park, Evidence that cisplatin-induced auditory damage is attenuated by downregulation of pro-inflammatory cytokines via Nrf2/HO-1, JARO—J. Assoc. Res. Otolaryngol. 9 (2008) 290-306. https://doi.org/10.1007/s10162-008-0126-y.
[26] M. Uy, J. K. Telford, Optimization by design of experiment techniques, in: IEEE Aerosp. Conf. Proc., 2009. https://doi.org/10.1109/AERO.2009.4839625.
[27] J. Antony, Design of Experiments for Engineers and Scientists, 2003. https://doi.org/10.1016/B978-0-7506-4709-0.X5000-5.
[28] P. Improvement, 5.1.3. What are the steps of DOE?, (2017) 12-13. https://www.itl.nist.gov/div898/handbook/pri/section1/pri13.htm (accessed Mar. 14, 2024).
[29] M. Proust, Design of Experiments Guide, Screening. (n.d.). https://www.jmp.com/support/help/en/17.2/index.shtml#page/jmp/introduction-to-doe.shtml (accessed Mar. 14, 2024).
[30] S. Bhattacharya, Central Composite Design for Response Surface Methodology and Its Application in Pharmacy, in: Response Surf. Methodol. Eng. Sci., IntechOpen, 2021. https://doi.org/10.5772/intechopen.95835.
[31] NIST SEMATECH, 5.3.3.6.1. Central Composite Designs (CCD), Eng. Stat. Handb. (2020). https://www.itl.nist.gov/div898/handbook/pri/section3/pri3361.htm (accessed March 14, 2024).
[32] D. Toms, R. Deardon, M. Ungrin, Climbing the mountain: Experimental design for the efficient optimization of stem cell bioprocessing, J. Biol. Eng. 11 (2017) 35. https://doi.org/10.1186/s13036-017-0078-z.
[33] N. O'Connor, M. Geary, M. Wharton, P. Sweetman, Development and validation of a rapid chromatographic method for the analysis of flunarizine and its main production impurities, J. Pharm. Anal. 3 (2013) 211-214. https://doi.org/10.1016/j.jpha.2012.12.005.
[34] L. Wang, W. Wu, L. Wang, L. Wang, X. Zhao, Highly water-soluble solid dispersions of honokiol: Preparation, solubility, and bioavailability studies and anti-tumor activity evaluation, Pharmaceutics. 11 (2019) 573. https://doi.org/10.3390/pharmaceutics11110573.
[35] ICH, International Conference on Harmonisation of Technical Requirements for Registration of Pharmaceuticals for Human Use ICH Harmonised Tripartite Guideline Validation of Analytical Procedures: Text and Methodology Q2(R1), Fed. Regist. Vol. 62, No. 96. (1997) 27463-7. www.somatek.com (accessed Sep. 6, 2023).
[36] M. Proust, Design of Experiments Guide, Screening. (n.d.). https://www.jmp.com/support/help/en/17.1/index.shtml #page/jmp/introduction-to-doe.shtml %23 (accessed Sep. 20, 2023).
[37] C. L. Kaiser, B. J. Chapman, J. L. Guidi, C. E. Terry, D. A. Mangiardi, D. A. Cotanche, Comparison of activated caspase detection methods in the gentamicin-treated chick cochlea, Hear. Res. 240 (2008) 1-11. https://doi.org/10.1016/j.heares.2008.03.003.
[38] J. Chen, R. Zhou, L. Li, B. Li, X. Zhang, J. Su, Mechanical, rheological and release behaviors of a poloxamer 407/poloxamer 188/carbopol 940 thermosensitive composite hydrogel, Molecules. 18 (2013) 12415-12425. https://doi.org/10.3390/molecules181012415.
[39] E. Quarta, M. Chiappi, A. Adamiano, A. Tampieri, W. Wang, T. D. Tetley, F. Buttini, F. Sonvico, D. Catalucci, P. Colombo, M. Iafisco, L. Degli Esposti, Inhalable Microparticles Embedding Biocompatible Magnetic Iron-Doped Hydroxyapatite Nanoparticles, J. Funct. Biomater. 14 (2023) 189. https://doi.org/10.3390/jfb14040189.
[40] R. W. Korsmeyer, R. Gurny, E. Doelker, P. Buri, N. A. Peppas, Mechanisms of solute release from porous hydrophilic polymers, Int. J. Pharm. 15 (1983) 25-35. https://doi.org/10.1016/0378-5173(83)90064-9.
[41] Y. Huang, X. Huang, S. Wang, Y. Yu, S. Ni, Q. Qin, Soft-shelled turtle iridovirus enters cells via cholesterol-dependent, clathrin-mediated endocytosis as well as macropinocytosis, Arch. Virol. 163 (2018) 3023-3033. https://doi.org/10.1007/s00705-018-3966-8.
[42] C. Godlee, M. Kaksonen, From uncertain beginnings: Initiation mechanisms of clathrin-mediated endocytosis, J. Cell Biol. 203 (2013) 717-725. https://doi.org/10.1083/jcb.201307100.
[43] X. Wang, Y. Zhou, D. Wang, Y. Wang, Z. Zhou, X. Ma, X. Liu, Y. Dong, Cisplatin-induced ototoxicity: From signaling network to therapeutic targets, Biomed. Pharmacother. 157 (2023) 114045. https://doi.org/10.1016/j.biopha.2022.114045.
[44] J. Wu, J. Ye, W. Kong, S. Zhang, Y. Zheng, Programmed cell death pathways in hearing loss: A review of apoptosis, autophagy and programmed necrosis, Cell Prolif. 53 (2020) e12915. https://doi.org/10.1111/cpr.12915.
[45] FDAApproval Marks New Era for Preventing Cisplatin-Induced Hearing Loss|Children's Hospital Los Angeles, (n.d.). https://www.chla.org/clinical/research-and-breakthroughs/fda-approval-marks-new-era-preventing-cisplatin-induced-hearing (accessed Jan. 19, 2024).
[46] A. Chattaraj, M. P. Syed, C. A. Low, T. K. Owonikoko, Cisplatin-Induced Ototoxicity: A Concise Review of the Burden, Prevention, and Interception Strategies, JCO Oncol. Pract. 19 (2023) 278-283. https://doi.org/10.1200/op.22.00710.
[47] X. Tan, Y. Zhou, A. Agarwal, M. Lim, Y. Xu, Y. Zhu, J. O'Brien, E. Tran, J. Zheng, D. Gius, C.-P. Richter, Systemic application of honokiol prevents cisplatin ototoxicity without compromising its antitumor effect, Am. J. Cancer Res. 10 (2020) 4416-4434. http://www.ncbi.nlm.nih.gov/pubmed/33415008%0Ahttp://www.pubmedcentral.nih.gov/articlere nder.fcgi?artid=PMC7783741 (accessed Dec. 18, 2023).
[48] H. S. So, H. J. Kim, J. H. Lee, J. H. Lee, S. Y. Park, C. Park, Y. H. Kim, J. K. Kim, K. M. Lee, K. S. Kim, S. Y. Chung, W. C. Jang, S. K. Moon, H. T. Chung, R. K. Park, Flunarizine induces Nrf2-mediated transcriptional activation of heme oxygenase-1 in protection of auditory cells from cisplatin, Cell Death Differ. 13 (2006) 1763-1775. https://doi.org/10.1038/sj.cdd.4401863.
[49] N. S. Thakur, I. Rus, E. Sparks, V. Agrahari, Dual stimuli-responsive and sustained drug delivery NanoSensoGel formulation for prevention of cisplatin-induced ototoxicity, J. Control. Release. 368 (2024) 66-83. https://doi.org/10.1016/J.JCONREL.2024.02.005.
[50] J. M. White, M. A. Calabrese, Impact of small molecule and reverse poloxamer addition on the micellization and gelation mechanisms of poloxamer hydrogels, Colloids Surfaces A Physicochem. Eng. Asp. 638 (2022) 128246. https://doi.org/10.1016/j.colsurfa.2021.128246.
[51] N. Hirun, P. Kraisit, V. Tantishaiyakul, Thermosensitive Polymer Blend Composed of Poloxamer 407, Poloxamer 188 and Polycarbophil for the Use as Mucoadhesive In Situ Gel, Polymers (Basel). 14 (2022) 1836. https://doi.org/10.3390/polym14091836.
[52] G. Choi, E. I. Fitriasari, C. Park, Electro-Mechanochemical Gating of a Metal-Phenolic Nanocage for Controlled Guest-Release Self-Powered Patches and Injectable Gels, ACS Nano. 15 (2021) 14580-14586. https://doi.org/10.1021/acsnano.1c04276.
[53] M. Fosca, J. V. Rau, V. Uskokovid, Factors influencing the drug release from calcium phosphate cements, Bioact. Mater. 7 (2022) 341-363. https://doi.org/10.1016/j.bioactmat.2021.05.032.

What is claimed is:

1. A method of mitigating or reducing the effects of a drug-induced ototoxicity or drug-induced hearing loss in a subject in need of such treatment, comprising: providing a drug delivery system which releases a therapeutic drug upon exposure to reactive oxygen species (ROS), wherein the drug delivery system comprises a thermoresponsive hydrogel (HY) containing therapeutic drug-loaded crosslinked hybrid nanoparticles (drug-cHy-NPs) comprising polypropylene sulfide-polyethylene glycol monomethyl ether (PPS-mPEG) crosslinked with 10,12-Pentacosadiynoic acid (PCDA), and wherein the drug-cHy-NPs provide sustained release of the therapeutic drug.

2. The method of claim 1, wherein the therapeutic drug is selected from the group consisting of Flunarizine (FL), Honokiol (HK), Amifostine, Sodium Thiosulfate (STS), STS-IV, Bucillamine, Diltiazem, Dexamethasone, N-acetylcysteine, Ebselen, Agmatine, and Allicin.

3. The method of claim 1, wherein in the NPs, the PCDA:PPS-mPEG ratio is in a range of about 1:2 w/w to about 3:5 w/w.

4. The method of claim 1, wherein the mPEG is mPEG₂₀₀₀.

5. The method of claim 1, wherein the drug-cHy-NPs provide sustained drug release at a temperature of about 37° C.

6. The method of claim 1, wherein the HY comprises a mixture of a poloxamer and a carbomer.

7. The method of claim 1, wherein the drug-induced ototoxicity or drug-induced hearing loss is induced by a platinum-based drug selected from the group consisting of cisplatin, carboplatin, oxaliplatin, nedaplatin, heptaplatin, lobaplatin, miriplatin, tetraplatin, iproplatin, satraplatin, ormaplatin, and oxoplatin.

8. A composition, comprising: a drug delivery system which releases a therapeutic drug upon exposure to reactive oxygen species (ROS), wherein the drug delivery system comprises a thermoresponsive hydrogel (HY) containing therapeutic drug-loaded crosslinked hybrid nanoparticles (drug-cHy-NPs) comprising polypropylene sulfide-polyethylene glycol monomethyl ether (PPS-mPEG) crosslinked with 10,12-Pentacosadiynoic acid (PCDA), and wherein the drug-cHy-NPs provide sustained release of the therapeutic drug.

9. The composition of claim 8, wherein the therapeutic drug is selected from the group consisting of Flunarizine (FL), Honokiol (HK), Amifostine, Sodium Thiosulfate (STS), STS-IV, Bucillamine, Diltiazem, Dexamethasone, N-acetylcysteine, Ebselen, Agmatine, and Allicin.

10. The composition of claim 8, wherein in the NPs, the PCDA:PPS-mPEG ratio is in a range of about 1:2 w/w to about 3:5 w/w.

11. The composition of claim 8, wherein the mPEG is mPEG₂₀₀₀.

12. The composition of claim 8, wherein the drug delivery system provides sustained therapeutic drug release at a temperature of about 37° C.

13. The composition of claim 8, wherein the HY comprises a mixture of a poloxamer and a carbomer.
